(19) United States
(12) Patent Application Publication
(10) Pub. No.: US 2026/0097224 A1
(43) Pub. Date: Apr. 9, 2026

(54) METHODS FOR MODULATING NEUROPLASTICITY

(71) Applicant: Ampa Inc., San Diego, CA (US)

(72) Inventors: Donald A. Vaughn, San Diego, CA (US); Jonathan A.S. Downar, Carrying Place (CA)

(21) Appl. No.: 19/274,159

(22) Filed: Jul. 18, 2025

Related U.S. Application Data
(60) Provisional application No. 63/703,576, filed on Oct. 4, 2024.

(51) Int. Cl. A61N 2/00; A61N 1/36

(52) U.S. Cl. CPC A61N 2/002; A61K 31/42; A61N 1/36; A61N 1/36025; A61N 2/006; A61P 25/28

(57) ABSTRACT

Disclosed is a method for providing targeted energy-based neurostimulation treatment of the brain of a patient in need of such treatment, which method involves administering to said patient prior to or simultaneously with administration of said targeted neurostimulation treatment sufficient amount of a dopamine receptor agonist in combination with one or more drugs that modulate or enhance neuroplasticity of the brain of the patient, and repeating said targeted neurostimulation treatment for a time period not exceeding one day. In a preferred embodiment the treatment is for depression, and the targeted neurostimulation treatment involves transcranial magnetic stimulation.

### CROSS REFERENCE TO RELATED APPLICATION

This application claims priority to U.S. Provisional Application Ser. No. 63/703,576, filed Oct. 4, 2024, the contents of which are incorporated herein by reference.

### FIELD OF THE DISCLOSURE

The present disclosure is generally related to treating brain disorders through neuromodulation techniques. The disclosure has particular utility for treating depression using brain stimulation such as transcranial magnetic stimulation (TMS) and will be described in connection with such utility, although other utilities are contemplated including treatment of other brain disorders such as anxiety, cognitive or behavioral disorders including but not limited to anger management, compulsive gambling, excessive smoking and/or drinking, eating disorders and the like, and various neurodegenerative disorders such as Parkinson's Disease, ALS and Huntington's Disease, or stroke using brain stimulation systems including TMS and other external brain stimulation systems such as particle-beam systems including gamma ray particles and electrons or photons from the electromagnetic spectrum including radio waves, x-rays, ultraviolet light, gamma rays, or focused ultrasound, which are given as exemplary but limitation.

### BACKGROUND OF THE DISCLOSURE

Decades of research have revealed that, for patients diagnosed with depression, specific brain regions are under-active. These areas normally help the brain regulate emotions. Strengthening these areas to normal levels can restore the brain's delicate balance of activity and lead to a dramatic improvement in mood. While there are several anti-depressant medications and psychological that may be effective, many patients are unable to achieve significant benefit from either medication or psychological treatment or their combination. Moreover, antidepressant medications oftentimes trigger undesirable side effects such as fatigue, dry mouth, emotional flatness, forgetfulness, weight gain, sweating, constipation, sexual dysfunction, insomnia and/or drowsiness. As a result, many patients discontinue treatment and/or may relapse after treatment, and/or become dependent on continuing treatment. Non-invasive repetitive TMS of brain regions associated with depression has emerged as a treatment option for individuals who have not benefited from or not tolerated traditional psychological and pharmacotherapy treatment. Rates of clinical response to non-invasive repetitive TMS are 33-50%. TMS offers an alternative and effective treatment without side effects or relapse of traditional psychological and pharmacotherapy treatment. TMS uses gentle magnetic pulses to strengthen a target part of the brain that is under-active. TMS involves multiple treatments over short durations of time. A typical treatment course may involve multiple, e.g., 36-50 short, e.g. 3-10 minute sessions given daily (e.g. Monday through Friday) over several weeks. After treatment, for responders, the brain region will come back into balance.

WO 2022/246557 discloses the administration to a patient undergoing treatment for major depression disorder (MDD) by TMS of a low dose NMDA receptor such as D-cycloserine (DCS). According to WO 2022/246557, DCS is provided as a daily dose just prior to or concurrent with daily (e.g. Monday through Friday) TMS treatment over several, e.g. four weeks. As taught by WO 2022/246557, low dose DCS include doses ranging from about 1-250 mg of DCS, resulting in 1-30 μg/mL plasma concentrations enhances TMS treatment effects and improves treatment outcomes for patients undergoing once-per-day treatments. However, committing to forty to fifty sessions five days a week over several weeks is difficult, particularly for busy people and is costly to administer in any event.

### SUMMARY OF THE DISCLOSURE

Disclosed in one aspect are methods for enhancing or modulating neuroplasticity through a combination of neuromodulation techniques and external interventions. The core approach involves a treatment protocol involving, using multiple sessions of brain stimulation technologies such as transcranial magnetic stimulation (TMS) or other external brain stimulation systems such as particle-beam systems including gamma ray particles and electrons or photons from the electromagnetic spectrum including radio waves, x-rays, ultrasonic and gamma rays, or focused ultrasound, neuroplasticity-potentiating pharmaceutical agents. Useful neuroplasticity-potentiating pharmaceutical agents are chemical agents that influence neurotransmitter systems, neurotrophic factors, calcium signaling, and cytoskeletal restructuring, offering novel approaches to neuroplasticity-modulation in neurological and psychiatric conditions. In one aspect a combination of neuroplasticity-potentiating agents that can both increase neuroplasticity in one region and inhibit it in another, potentially addressing unwanted side effects may be used. The multiple neuromodulation treatment sessions should be delivered to the patient during a time period the neuroplasticity-potentiating pharmaceutical agent remains pharmacologically active.

In a preferred aspect of the disclosure the neuromodulation treatment sessions are administered to a patient during a time period not exceeding five half-life periods of the neuroplasticity-potentiating pharmaceutical agent.

These disclosed methods are applicable for treating a range of conditions, including depression, anxiety, cognitive disorders, and neurodegenerative disorders, making the subject application a comprehensive framework for modulating brain plasticity across multiple domains.

More particularly, and as applied to the treatment of depression employing TMS, we have found that we can significantly reduce the total time spent delivering treatment, including rest periods, from several weeks to a single work day, by enhancing or modulating the neuroplasticity of the brain of a patient by administering to the patient prior to, with contemporaneously or following commencement of delivery of energy to the brain of the patient using TMS, certain drugs and drug combinations. Particularly useful drugs include various selected pharmaceutical agents selected to potentiate the neuromodulation effects of delivery of energy as will be described below. Administration of a selected pharmaceutical agents prior to, simultaneously with or following commencement of delivery of energy to the brain of the patient permits us to reduce the number and duration of treatment sessions and rest periods so that the total time spent delivering of the treatment including rest periods is significantly reduced to a single work day. For example, in the case of TMS treatment for depression, rather than 36 short, e.g., 3-10 minute treatments given daily over several weeks, a full treatment protocol in accordance with the present disclosure may involve 20 short 3-10 minute treatments given over a single work day, e.g., of 12 hours or less. Another feature and advantage of enhancing or modulating the neuroplasticity of the brain of a patient by administering prior to or contemporaneously with delivery of energy to the brain of the patient is using TMS, certain drugs and drug combinations in accordance with the present disclosure, is that it is less critical to essentially exactly and repeatedly hit the same target area of the brain over multiple treatments.

The process of the present disclosure may be used for treating a range of conditions, including depression, anxiety, cognitive disorders, and various neurodegenerative diseases, which conditions may be benefit from modulating brain plasticity.

In one aspect the disclosure provides a method for providing targeted neurostimulation treatment of the brain of a patient in need of such treatment, comprising administering to said patient prior to or simultaneously with or following administration of said targeted neurostimulation treatment a sufficient amount of a selected pharmacological agent selected to potentiate the neuromodulatory effects of the neurostimulation treatment, and repeating said targeted neurostimulation treatment for a time period while the selected pharmaceutical agent remains pharmacologically active. Typically a single dose of a selected pharmacological agent that potentiates the neuromodulatory effects of neurostimulation is administered to a patient prior to, with or following commencement of delivery of energy to the brain patient, and multiple sessions of delivery of energy to the brain are provided during the period of pharmacological activity of the medication. In the case of medications having a half-life of 8 hours or more, one dose typically will be sufficient so that the entire treatment sessions can be completed in a single work day. If treatment is spread over two days for the convenience of the patient, or as needed, a second dose of the pharmaceutical may be administered. Alternatively, partial doses of the pharmaceutical may be administered during sessions of delivery of energy to the brain.

Various selected pharmacological agents that potentiate the neuromodulatory effects of neurostimulation advantageously may be employed including, by way of example, but not limitation, dopaminergic pharmacological agents, glutamatergic agents (excluding N-methyl-D-aspartate receptors (NMDA receptors)), and combinations of dopaminergic pharmacological agents and NMDAergic pharmaceutical agents.

In one embodiment the dopaminergic pharmacological agent is administered in combination or sequentially with one or more exogenous drugs that influence dopamine levels of the patient.

In another embodiment the dopaminergic pharmacological agent is administered in combination or sequentially with one or more exogenous drugs that influence the cholinergic system of the patient.

In a further embodiment the dopaminergic pharmacological agent is administered in combination or sequentially with one or more exogenous drugs that influence glutamatergic system of the patient.

In still yet another embodiment the dopaminergic pharmacological agent is administered in combination or sequentially with one or more exogenous drugs that influence NMDA receptor activity of the patient.

In a further embodiment the dopaminergic pharmacological agent is administered in combination or sequentially with one or more exogenous drugs that modify calcium-based plasticity of the patient.

In another embodiment the dopaminergic pharmacological agent is administered in combination or sequentially with one or more exogenous drugs that modify cytoskeletal plasticity of the patient.

In a still further embodiment the dopaminergic pharmacological agent is administered in combination or sequentially with one or more exogenous drugs that modify chromatin restructuring of the patient.

In yet another embodiment the dopaminergic pharmacological agent is administered in combination or sequentially with one or more exogenous drugs that modify molecular signaling pathways of the patient.

In another embodiment the dopaminergic pharmacological agent is administered in combination or sequentially with one or more exogenous drugs that modify structural plasticity of the patient.

In yet another embodiment the dopaminergic pharmacological agent is administered in combination or sequentially with one or more exogenous drugs that influence neurotrophic factors of the patient.

In a further embodiment the dopaminergic pharmacological agent is administered in combination or sequentially with one or more exogenous drugs that influence glial cell activity of the patient.

In a particularly preferred embodiment the targeted neurostimulation treatment comprises transcranial magnetic stimulation (TMS).

In a further embodiment the targeted neurostimulation treatment comprises treatment with a particle-beam, gamma ray particles, electrons, protons, radio waves, x-rays, ultraviolet light, gamma rays, or focused ultrasound.

In another and preferred embodiment the treatment is for depression.

In yet another embodiment the treatment is for major depressive disorder and/or anxiety, or other psychiatric disorders such as: bipolar disorder; obsessive-compulsive disorder; post-traumatic stress disorder; other anxiety disorders (including but not limited to generalized anxiety disorder, panic disorder, social anxiety disorder, or specific phobias such as fear of flying); attention deficit/hyperactivity disorder; Tourette syndrome, body-focused repetitive behaviors; substance abuse or dependence; sleep disorders including primary or secondary insomnia; eating disorders such as anorexia nervosa, bulimia nervosa, or binge eating disorder; impulse control disorders such as pathological gambling; psychotic disorders such as schizophrenia, schizophreniform disorder; autism spectrum disorders; personality disorders such as borderline personality disorder or obsessive-compulsive personality disorder.

In still another embodiment the treatment is for a neurodegenerative disorder, such as Alzheimer's disease, Parkinson's disease, Huntington's disease, corticobasal degeneration, spinocerebellar ataxia, amyotrophic lateral sclerosis, frontotemporal dementia, chronic traumatic encephalopathy, or post-stroke neurodegeneration, or for other neurological disorder such as tinnitus, migraine, stroke, epilepsy, multiple sclerosis, or chronic pain

More particularly, in accordance with Aspect A, there is provided a method for enhancing and/or accelerating the therapeutic effect of a neurostimulation treatment of the brain of an individual, wherein said neuromodulation treatment protocol comprising multiple sessions delivered over a period of time, comprising administering to the patient prior to, concurrently with, or following commencement of said administration of said neuromodulation treatment, a neuroplasticity-potentiating pharmaceutical agent, whereupon the neuromodulating potentiating agent comprises D-cycloserine (DCS) or a N-methyl-D-aspartate receptor agonist (NMDA), and the multiple neuromodulation treatment sessions are delivered to the patient during a time period the neuroplasticity-potentiating pharmaceutical agent remains pharmacologically active.

In one embodiment of Aspect A, the neurostimulation sessions are administered during a time period not exceeding five half-life periods of the neuroplasticity potentiating pharmaceutical agent.

In another embodiment of Aspect A, the neurostimulation treatment comprises transcranial magnetic stimulation (TMS).

In yet another embodiment of Aspect A, the neurostimulation treatment comprises transcranial electrical stimulation of the brain via a non-convulsive technique such as transcranial direct current stimulation (tDCS), transcranial alternating current stimulation (tACS), transcranial random noise stimulation, (tRNS), transcranial temporal interference stimulation (tTIS), or deep brain stimulation (DBS), or minimally invasive bioelectric implantation.

In a further embodiment of Aspect A, the neurostimulation treatment comprises a convulsive technique such as electroconvulsive therapy (ECT) or magnetic seizure therapy (MST).

In a still further embodiment of Aspect A, the targeted neurostimulation treatment comprises treatment with transcranial focused ultrasound (FUS) or transcranial photobiomodulation.

In a particularly preferred further embodiment of Aspect A, the treatment is for major depressive disorder and/or anxiety.

According to Aspect B, there is provided a method for enhancing and/or accelerating the therapeutic effect of a neurostimulation treatment of the brain of an individual, wherein said neuromodulation treatment protocol comprising multiple sessions delivered over a period of time, comprising administering to the patient prior to, concurrently with, or following commencement of said administration of said neuromodulation treatment, a neuroplasticity-potentiating pharmaceutical agent, whereupon the neuroplasticity potentiating pharmaceutical agent is selected from the group consisting of a non-NMDA glutamatergic pharmacological agent, a dopaminergic pharmacological agent and an NMDAergic pharmacological agent, a cholinergic agent, a calcium-mediated plasticity agent, a cytoskeletal restructuring agent, a chromatin remodeling agent, a synaptic reorganization agent and a neurotropic agent.

In one embodiment of Aspect B, the neuroplasticity-potentiating agent is selected from the group consisting of dopaminergic pharmacological agent, preferably a dopamine precursor agent, more preferably L-DOPA (Levodopa); a dopamine agonist, preferably Pramipexole, Ropinirole, Bromocriptine, Quinpirole, Rotigotine, or Apomorphine; a monoamine oxidase inhibitor (MAOI), preferably Selegiline, Rasagiline, Phenelzine, or Tranylcypromine; a catechol-O-methyltransferase (COMT) inhibitor, preferably Entacapone, Tolcapone, or Opicapone; a dopamine reuptake inhibitor (DRI), preferably Bupropion, Nomifensine, Amineptine, or Tianeptine; a combined dopaminergic therapy, preferably Sinemet (Levodopa/Carbidopa) or Stalevo (Levodopa/Carbidopa/Entacapone); a stimulant increasing dopamine levels, preferably Amphetamine, Dextroamphetamine, Mixtures of Dextroamphetamine and Levoamphetamine, Lisdexamfetamine, Methamphetamine, Methylphenidate, or Dexmethylphenidate; and an adenosine receptor blocker, preferably Caffeine, Theophylline, Theobromine, Istradefylline, or Preladenant.

In another embodiment of Aspect B, the neurostimulation sessions are administered during a time period the neuroplasticity-potentiating pharmaceutical agent remains pharmacologically active.

In still another embodiment of Aspect B, the neurostimulation sessions are administered during a time period not exceeding five half-life periods of the neuroplasticity potentiating pharmaceutical agent.

In a further embodiment of Aspect B, the neuroplasticity-potentiating agent comprises a non-NMDA glutamatergic pharmacological agent, selected from the group consisting of an AMPA receptor modulator, preferably CX-516, CX-614, Aniracetam, Sunifiram, Piracetam, Pramiracetam, or Perampanel; an AMPA receptor agonist, preferably Ibotenic Acid or Quisqualic Acid; a kainate receptor modulator, preferably Topiramate, LY466195, or NS102; a metabotropic glutamate receptor (mGluR) modulator, preferably LY341495, Mavoglurant, LY354740, or MPEP; a glutamate release modulator, preferably Lamotrigine or Riluzole; a glutamate reuptake modulator, preferably Ceftriaxone; and a vesicular glutamate transporter (VGLUT) modulator, preferably Furosemide.

In yet another embodiment of Aspect B, the neuroplasticity-potentiating agent comprises a dopaminergic pharmacological agent and an NMDAergic pharmacological agent selected from the group consisting of an NMDA receptor agonist or partial agonist, preferably Cycloserine, D-Cycloserine, Rapastinel, Apimostinel, or Zelquistinel; a glycine site modulator (NR1 co-agonist), preferably Glycine, D-Serine, D-Alanine, or Sarcosine; and a polyamine site modulator, preferably Ifenprodil or Eliprodil.

In a further embodiment of Aspect B, the neurostimulation treatment comprises transcranial magnetic stimulation (TMS).

In another embodiment of Aspect B, the neurostimulation treatment comprises transcranial electrical stimulation of the brain via a non-convulsive technique such as transcranial direct current stimulation (tDCS), transcranial alternating current stimulation (tACS), transcranial random noise stimulation, (tRNS), transcranial temporal interference stimulation (tTIS), or deep brain stimulation (DBS), or minimally invasive bioelectric implantation.

In yet another embodiment of Aspect B, the neurostimulation treatment comprises a convulsive technique such as electroconvulsive therapy (ECT) or magnetic seizure therapy (MST).

In another embodiment of Aspect B, the targeted neurostimulation treatment comprises treatment with transcranial focused ultrasound (FUS) or transcranial photobiomodulation.

In a further embodiment of Aspect B, the treatment is for major depressive disorder and/or anxiety.

In yet another embodiment of Aspect B, the treatment is for a psychiatric disorders preferably: bipolar disorder; obsessive-compulsive disorder; post-traumatic stress disorder; other anxiety disorders (including but not limited to generalized anxiety disorder, panic disorder, social anxiety disorder, or specific phobias such as fear of flying); attention deficit/hyperactivity disorder; Tourette syndrome, body-focused repetitive behaviors; substance abuse or dependence; sleep disorders including primary or secondary insomnia; eating disorders such as anorexia nervosa, bulimia nervosa, or binge eating disorder; impulse control disorders such as pathological gambling; psychotic disorders such as schizophrenia, schizophreniform disorder; autism spectrum disorders; or a personality disorder such as borderline personality disorder or obsessive-compulsive personality disorder.

In still another embodiment of Aspect B, the treatment is for a neurodegenerative disorder selected from the group consisting of Alzheimer's disease, Parkinson's disease, Huntington's disease, corticobasal degeneration, spinocerebellar ataxia, amyotrophic lateral sclerosis, frontotemporal dementia, chronic traumatic encephalopathy, and post-stroke neurodegeneration.

In yet another embodiment of Aspect B, the treatment is for a neurological disorder selected from the group consisting of tinnitus, migraine, stroke, epilepsy, multiple sclerosis, and chronic pain.

According to Aspect C, there is provided a method for enhancing and/or accelerating the therapeutic effect of a neurostimulation treatment of the brain of an individual, wherein said neuromodulation treatment protocol comprising multiple sessions delivered over a period of time, comprising administering to the patient prior to, concurrently with, or following commencement of said administration of said neuromodulation treatment, a neuroplasticity-potentiating pharmaceutical agent, whereupon the neuroplasticity potentiating pharmaceutical agent comprises A) D-cycloserine (DCS), a N-methyl-D-aspartate receptor agonist (NMDA), or DCS and NMDA, and B) a dopaminergic pharmacological agent, a non-NMDA glutamatergic pharmacological agent, a dopaminergic pharmacological agent and an NMDAergic pharmacological agent, a cholinergic agent, a calcium-mediated plasticity agent, a cytoskeletal restructuring agent, a chromatin remodeling agent, a synaptic reorganization agent or a neurotropic agent.

In one embodiment of Aspect C, the neuroplasticity-potentiating agent is selected from the group consisting of dopaminergic pharmacological agent, preferably a dopamine precursor agent, more preferably L-DOPA (Levodopa); a dopamine agonist, preferably Pramipexole, Ropinirole, Bromocriptine, Quinpirole, Rotigotine, or Apomorphine; a monoamine oxidase inhibitor (MAOI), preferably Selegiline, Rasagiline, Phenelzine, or Tranylcypromine; a catechol-O-methyltransferase (COMT) inhibitor, preferably Entacapone, Tolcapone, or Opicapone; a dopamine reuptake inhibitor (DRI), preferably Bupropion, Nomifensine, Amineptine, or Tianeptine; a combined dopaminergic therapy, preferably Sinemet (Levodopa/Carbidopa) or Stalevo (Levodopa/Carbidopa/Entacapone); a stimulant increasing dopamine levels, preferably Amphetamine, Dextroamphetamine, Mixtures of Dextroamphetamine and Levoamphetamine, Lisdexamfetamine, Methamphetamine, Methylphenidate, or Dexmethylphenidate; and an adenosine receptor blocker, preferably Caffeine, Theophylline, Theobromine, Istradefylline, or Preladenant.

In another embodiment of Aspect C, the neurostimulation sessions are administered during a time period the neuroplasticity-potentiating pharmaceutical agent remains pharmacologically active.

In a further embodiment of Aspect C, the neurostimulation sessions are administered during a time period not exceeding five half-life periods of the neuroplasticity potentiating pharmaceutical agent.

In yet another embodiment of Aspect C, the neuroplasticity-potentiating agent comprises a non-NMDA glutamatergic pharmacological agent, selected from the group consisting of an AMPA receptor modulator, preferably CX-516, CX-614, Aniracetam, Sunifiram, Piracetam, Pramiracetam, or Perampanel; an AMPA receptor agonist, preferably Ibotenic Acid or Quisqualic Acid; a kainate receptor modulator, preferably Topiramate, LY466195, or NS102; a metabotropic glutamate receptor (mGluR) modulator, preferably LY341495, Mavoglurant, LY354740, or MPEP; a glutamate release modulator, preferably Lamotrigine or Riluzole; a glutamate reuptake modulator, preferably Ceftriaxone; and a vesicular glutamate transporter (VGLUT) modulator, preferably Furosemide.

In still yet another embodiment of Aspect C, the neuroplasticity-potentiating agent comprises a dopaminergic pharmacological agent and an NMDAergic pharmacological agent selected from the group consisting of an NMDA receptor agonist or partial agonist, preferably Cycloserine, D-Cycloserine, Rapastinel, Apimostinel, or Zelquistinel; a glycine site modulator (NR1 co-agonist), preferably Glycine, D-Serine, D-Alanine, or Sarcosine; and a polyamine site modulator, preferably Ifenprodil or Eliprodil.

In a preferred embodiment of Aspect C, the neurostimulation treatment comprises transcranial magnetic stimulation (TMS).

In another embodiment of Aspect C, the neurostimulation treatment comprises transcranial electrical stimulation of the brain via a non-convulsive technique such as transcranial direct current stimulation (tDCS), transcranial alternating current stimulation (tACS), transcranial random noise stimulation, (tRNS), transcranial temporal interference stimulation (tTIS), or deep brain stimulation (DBS), or minimally invasive bioelectric implantation.

In a further embodiment of Aspect C, the neurostimulation treatment comprises a convulsive technique such as electroconvulsive therapy (ECT) or magnetic seizure therapy (MST).

In another embodiment of Aspect C, the targeted neurostimulation treatment comprises treatment with transcranial focused ultrasound (FUS) or transcranial photobiomodulation.

In yet another embodiment of Aspect C, the treatment is for major depressive disorder and/or anxiety.

In a further embodiment of Aspect C, the treatment is for a psychiatric disorders preferably: bipolar disorder; obsessive-compulsive disorder; post-traumatic stress disorder; other anxiety disorders (including but not limited to generalized anxiety disorder, panic disorder, social anxiety disorder, or specific phobias such as fear of flying); attention deficit/hyperactivity disorder; Tourette syndrome, body-focused repetitive behaviors; substance abuse or dependence; sleep disorders including primary or secondary insomnia; eating disorders such as anorexia nervosa, bulimia nervosa, or binge eating disorder; impulse control disorders such as pathological gambling; psychotic disorders such as schizophrenia, schizophreniform disorder; autism spectrum disorders; or a personality disorder such as borderline personality disorder or obsessive-compulsive personality disorder.

In yet a further embodiment of Aspect C, the treatment is for a neurodegenerative disorder selected from the group consisting of Alzheimer's disease, Parkinson's disease, Huntington's disease, corticobasal degeneration, spinocerebellar ataxia, amyotrophic lateral sclerosis, frontotemporal dementia, chronic traumatic encephalopathy, and post-stroke neurodegeneration.

In still yet another embodiment of Aspect C, the treatment is for a neurological disorder selected from the group consisting of tinnitus, migraine, stroke, epilepsy, multiple sclerosis, and chronic pain.

More particularly, in accordance with Aspect D there is provided a method for enhancing and/or accelerating a therapeutic effect of an energy-based neuromodulation treatment of the central nervous system (CNS) in an individual to treat a condition selected from the group consisting of an anxiety disorder; a stress disorder; a psychotic disorder, a personality disorder; an impulse-control disorder; an addiction disorder; an eating disorder; a sleep disorder; a sexual disorder, a neurodevelopmental disorder; a neurological disorder; and a functional performance domain; the method comprising administering to the individual at least one neuroplasticity-potentiating pharmaceutical agent, and delivering said energy-based neuromodulation treatment of said individual within a time period less than five half-life time periods before, during or after of at least one of the neuroplasticity-potentiating pharmaceutical agent.

In one embodiment of Aspect D the energy-based neuromodulation treatment comprises at least one treatment method selected from the group consisting of transcranial magnetic stimulation (TMS); transcranial electrical stimulation; convulsive neuromodulation; energy-based focal stimulation; implantable bioelectric stimulation; and genetically-targeted neuromodulation.

In another embodiment of Aspect D the neuroplasticity-potentiating pharmaceutical agent comprises at least one agent selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator.

In a further embodiment of Aspect D the energy-based neuromodulation treatment comprises transcranial magnetic stimulation (TMS), and wherein the neuroplasticity-potentiating pharmaceutical agent comprises an NMDA receptor agonist, optionally administered in combination with a dopaminergic agent.

In still yet another embodiment of Aspect D characterized by one or more of the following conditions: (a) said energy-based neuromodulation treatment is predominantly delivered at less than 80% of an individualized therapeutic threshold; (b) a dominant pulse frequency for TMS is 5 Hz or less; (c) a onset-aligned inter-session interval is 30 minutes or less; and (d) a total number of therapeutic pulses per session is fewer than 600. As used herein the term “predominantly delivered” means 50% of said energy-based neuromodulation treatment is delivered at less than 80% of an individualized therapeutic threshold.

According to Aspect E, there is provided a method for enhancing and/or accelerating a therapeutic effect of an energy-based neuromodulation treatment of the central nervous system (CNS) in an individual, wherein said energy-based neuromodulation treatment comprises at least one neuromodulation treatment method selected from the group consisting of a transcranial electrical stimulation method; a convulsive neuromodulation method; an energy-based focal stimulation method; an implantable bioelectric stimulation method; and a genetically-targeted neuromodulation method, the method comprising administering to said individual at least one neuroplasticity-potentiating pharmaceutical agent, and delivering said energy-based neuromodulation treatment to said individual within a time period less than five half-life time periods before, during or after administration of said neuroplasticity-potentiating pharmaceutical agent.

In one embodiment of Aspect E the energy-based neuromodulation treatment is configured to target at least one condition selected from the group consisting of an anxiety disorder; a stress disorder; a psychotic disorder; a personality disorder; an impulse-control disorder; an addiction disorder; an eating disorder; a sleep disorder, a sexual disorder; a neurodevelopmental disorder; a neurological disorder; a functional performance domain; a major depressive disorder; and an obsessive-compulsive disorder.

In another embodiment of Aspect E the neuroplasticity-potentiating pharmaceutical agent comprises at least one pharmaceutical agent selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; structural modulator; a synaptic modulator; a neurotrophic modulator; a neuroimmune modulator; and a glial modulator.

In still yet another embodiment of Aspect E characterized by one or more of the following conditions: (a) said energy-based neuromodulation treatment is predominantly delivered at less than 80% of an individualized therapeutic threshold; (b) a dominant pulse frequency for TMS is 5 Hz or less; (c) an onset-aligned inter-session interval is 30 minutes or less; and (d) a total number of therapeutic pulses per session is fewer than 600. As used herein the term “predominantly delivered” means 50% of said energy-based neuromodulation treatment is delivered at less than 80% of an individualized therapeutic threshold.

According to Aspect F, there is provided a method for enhancing and/or accelerating a therapeutic effect of an energy-based neuromodulation treatment of the central nervous system (CNS) in an individual in need of said treatment, the method comprising administering to said individual within a time period less than five half-life time periods before, during or after delivery of said energy-based neuromodulation treatment, at least one neuroplasticity potentiating pharmaceutical agent selected from the group consisting of a non-N-methyl-D-aspartate (NMDA) glutamatergic modulator; a monoaminergic modulator; a cholinergic modulator; a gamma-aminobutyric acid (GABAergic) modulator; an opioidergic modulator, an endocannabinoid modulator; a cellular signaling modulator; an epigenetic modulator; a transcriptional modulator; a structural modulator; a synaptic modulator; a neurotrophic modulator; a neuroimmune modulator; and a glial modulator.

In one embodiment of Aspect F, energy-based neuromodulation treatment is configured to target at least one condition selected from the group consisting of an anxiety disorder; a stress disorder; a psychotic disorder; a personality disorder; an impulse-control disorder; an addiction disorder; an eating disorder; a sleep disorder; a sexual disorder; a neurodevelopmental disorder; a neurological disorder; a functional performance domain; a major depressive disorder; and an obsessive-compulsive disorder.

In another embodiment of Aspect F energy-based neuromodulation treatment comprises at least one method selected from the group consisting of transcranial magnetic stimulation (TMS); transcranial electrical stimulation; convulsive neuromodulation; energy-based focal stimulation; implantable bioelectric stimulation; and genetically-targeted neuromodulation.

In a further embodiment of Aspect F the (a) said energy-based neuromodulation treatment is predominantly delivered at less than 80% of an individualized therapeutic threshold; (b) a dominant pulse frequency is 5 Hz or less; (c) an onset-aligned inter-session interval is 30 minutes or less; and (d) a total number of therapeutic pulses per session is fewer than 600. As used herein the term “predominantly delivered” means 50% of said energy-based neuromodulation treatment is delivered at less than 80% of an individualized therapeutic threshold.

In accordance with Aspect G there is provided a method for enhancing and/or accelerating a therapeutic effect of transcranial magnetic stimulation (TMS) treatment of the central nervous system (CNS) of an individual for at least one condition selected from the group consisting of major depressive disorder, obsessive-compulsive disorder, and suicidal ideation, comprising administering to said individual an N-methyl-D-aspartate (NMDA) receptor agonist, optionally in combination with at least one additional neuroplasticity-potentiating pharmaceutical agent, before, during or after delivering of said TMS treatment, wherein a majority of said TMS treatment pulses are delivered over multiple sessions over a period less than or equal to three treatment days. As used herein “a majority of said TMS treatment” means that at least 80% of said TMS treatment is provided within the specified time period.

In one embodiment of Aspect G the additional neuroplasticity-potentiating pharmaceutical agent comprises at least one pharmaceutical agent selected from the groups consisting of an NMDA receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator.

In another embodiment of Aspect G characterized by one or more of the following conditions: (a) said TMS is delivered at less than 80% of an individualized therapeutic threshold; (b) the dominant pulse frequency for TMS is 5 Hz or less; (c) the onset-aligned inter-session interval is 30 minutes or less; and (d) the total number of therapeutic pulses per session is fewer than 600.

In accordance with Aspect H there is provided a method for imparting a therapeutic effect of an energy-based neuromodulation treatment of the central nervous system (CNS) in an individual, the method comprising delivering at least one neuromodulation treatment method selected from the group consisting of transcranial magnetic stimulation (TMS); transcranial electrical stimulation; convulsive neuromodulation; energy-based focal stimulation; implantable bioelectric stimulation; and genetically-targeted neuromodulation; wherein at least seventy-five percent of the total neuromodulatory energy is delivered within a continuous period of twelve hours or less, optionally further comprising administering at least one neuroplasticity-potentiating pharmaceutical agent, and, when such agent is administered, delivering said energy-based neuromodulation treatment within a time period less than five half-life time periods before, during, or after administration of the agent.

### BRIEF DESCRIPTION OF THE DRAWINGS

Further features and advantages of the disclosure will be seen in the following detailed description, taken in conjunction with the accompanying drawings. The drawings described herein are for illustrative purposes only of selected embodiments and not all possible implementations and are not intended to limit the scope of the present disclosure.

FIG. 1 plots mean HRSD-17 scores for single location treatment over twenty six weeks;

FIG. 2 plots HRSD-17 scores for triple location treatment over twelve weeks;

FIG. 3 plots HAM-A scores of twelve weeks;

FIG. 4 plots HRSD-17 scores for those with Bipolar I Disorder over five weeks; and

FIG. 5 plots HRSD-17 scores for double location treatment over 6 weeks.

### DETAILED DESCRIPTION OF THE DISCLOSURE

As used herein the term “Therapeutic Effect” refers broadly to treatment, management, alleviation, reduction, prevention, maintenance, improvement, enhancement, optimization, modulation, or prophylaxis of symptoms, disorders, conditions, functions, states, traits, or domains outlined herein. It explicitly includes ongoing maintenance, relapse prevention, symptom reduction, functional restoration, enhancement of quality of life, and improvements in general health and wellness outcomes.

The term “Disorder” or “Disorders” included

### 1. Anxiety and Stress Disorders

Generalized anxiety disorder (GAD)
PTSD, acute stress disorder
Panic disorder, phobias, selective mutism
Adjustment disorders, prolonged grief disorder
Bereavement-related disorders
Rumination, worry, stress resilience, adaptability
Postpartum anxiety
Hyperarousal, hypervigilance
Avoidance behaviors, withdrawal, isolation

### 2. Psychotic and Personality Disorders

Schizophrenia, schizoaffective disorder, delusional disorder, schizophreniform disorder
Bipolar disorder (including bipolar I disorder, bipolar II disorder, cyclothymic disorder, and bipolar disorder with psychotic features)
Borderline, antisocial, narcissistic, avoidant, obsessive-compulsive personality disorders
Dissociative identity disorder, depersonalization/derealization disorder
Somatic symptom disorder, illness anxiety disorder, conversion disorder
Social cognition deficits, impaired interpersonal functioning
Hallucinations (auditory, visual), paranoia, delusional thinking
Emotional blunting, emotional numbness, maladaptive coping strategies
Impaired judgment, impaired insight

### 3. Impulse-Control and Addiction Disorders

Substance abuse/dependence, pathological gambling
Intermittent explosive disorder, kleptomania, pyromania
Body-focused repetitive behaviors (skin-picking, hair-pulling)
Impulsivity, compulsivity, aggression, risk-taking behaviors, impaired impulse control

### 4. Eating, Sleeping, and Sexual Disorders

Anorexia nervosa, bulimia nervosa, binge eating disorder, ARFID
Insomnia, hypersomnia, narcolepsy, sleep apnea, restless legs syndrome, sleep paralysis
Sleep architecture disturbances, circadian rhythm dysfunction
Gender dysphoria, premenstrual dysphoric disorder

### 5. Neurodevelopmental Disorders

Autism spectrum disorders (ASD), ADHD, Tourette syndrome and tic disorders
Intellectual disability, learning disorders, speech/language disorders
Developmental coordination disorders, cognitive impairments
Creative thinking deficits, ideational flexibility, mental agility deficits
Repetitive behaviors, stereotyped and habitual behaviors

### 6. Neurological Disorders

Alzheimer's disease, Parkinson's disease, ALS, dementia variants, Huntington's disease
Dementia with Lewy bodies, frontotemporal dementia, vascular dementia, corticobasal degeneration
Progressive supranuclear palsy (PSP), chronic traumatic encephalopathy (CTE)
Spinocerebellar ataxia, Friedreich's ataxia, leukodystrophies and related myelin disorders
Epilepsy, seizure disorders, migraine/headache disorders (cluster, tension-type), stroke, TBI, concussion, post-concussion syndrome
Post-stroke neurodegeneration, multiple system atrophy (MSA), normal pressure hydrocephalus (NPH)
Multiple sclerosis (MS), peripheral neuropathy, chronic neuropathic pain
Chronic inflammatory demyelinating polyneuropathy (CIDP), Guillain-Barré syndrome (GBS)
Myasthenia gravis, motor neuron diseases (primary lateral sclerosis, progressive muscular atrophy)
Movement disorders (essential tremor, dystonia, chorea)
Neuromuscular disorders (muscular dystrophy, myotonic dystrophy, Charcot-Marie-Tooth disease, myopathies)
Vertigo, tinnitus, auditory and visual processing disorders, balance disorders
Spinal cord injury and spinal disorders, cerebral palsy
Neurogenic bowel and bladder dysfunction
Neurovascular disorders (cerebral aneurysm, arteriovenous malformation (AVM), cerebral venous thrombosis, subarachnoid hemorrhage, CNS vasculitis)
Dysautonomia (including postural orthostatic tachycardia syndrome (POTS))
Chronic fatigue syndrome/myalgic encephalomyelitis (CFS/ME)
Functional neurological disorders (FND)
Neurofibromatosis, hydrocephalus
Neuroinflammatory and autoimmune disorders (encephalitis, transverse myelitis, NMOSD, acute disseminated encephalomyelitis (ADEM))
Neuro-oncological disorders (brain tumors, spinal cord tumors, neurofibromatosis-related tumors)
Genetic and metabolic neurological disorders (Wilson's disease, phenylketonuria (PKU), Tay-Sachs disease, mitochondrial disorders)
Neuroendocrine and neuroimmune interactions (hypothalamic and pituitary disorders affecting neurological function)
Chronic pain syndromes (fibromyalgia, complex regional pain syndrome (CRPS))

### 7. Functional Performance Domains

Sensory processing, perception, sensory acuity, sensory integration
Motor coordination, motor function, movement initiation, motor learning
Cognitive function, cognition, memory, learning, attention, vigilance, sustained focus
Executive function, cognitive flexibility, problem-solving, decision-making
Energy, alertness, arousal, motivation, initiative
Emotional regulation, emotional resilience
Dissociation, craving, urge regulation
Social functioning, empathy, theory of mind, interpersonal effectiveness
Emotional well-being, psychological flourishing, resilience
Self-awareness, self-regulation, insight, self-control
Pleasure, interest, reward sensitivity (anhedonia reversal)
Prevention of relapse, maintenance of remission, prophylaxis
Enhancement, optimization, or maintenance of these domains in healthy or at-risk individuals

### 8. Major Depressive Disorder (MDD)

Unipolar depression, treatment-resistant depression (TRD)
Melancholic, atypical, psychotic, catatonic, mixed features depression
Seasonal affective disorder (SAD)
Depression with anxious distress
Postpartum depression
Depression associated with medical conditions (e.g., stroke, cancer, chronic pain)
Depression associated with neurological conditions (e.g., dementia, Parkinson's disease)
Depression characterized by cognitive impairment, fatigue, anhedonia, emotional numbness
Maintenance therapy, relapse prevention in depression

### 9. Obsessive-Compulsive Disorder (OCD)

OCD characterized by contamination fears, checking behaviors, symmetry obsessions
OCD characterized by intrusive thoughts, scrupulosity, taboo obsessions
OCD with hoarding compulsions
OCD with predominantly obsessional or predominantly compulsive symptoms
Pediatric-onset OCD
OCD associated with neurological disorders (e.g., Tourette syndrome, tic disorders, autism spectrum disorders)
OCD spectrum disorders, obsessive-compulsive related behaviors
Maintenance therapy, relapse prevention in OCD

The term “energy-based neuromodulation treatment” includes
Transcranial magnetic stimulation (TMS)
Transcranial electrical stimulation:
   Transcranial direct current stimulation (tDCS)
   Transcranial alternating current stimulation (tACS)
   Transcranial random noise stimulation (tRNS)
   Transcranial temporal interference stimulation (tTIS)
Convulsive neuromodulation:
   Electroconvulsive therapy (ECT)
   Magnetic seizure therapy (MST)
Energy-based focal stimulation:
   Transcranial focused ultrasound (FUS)
   Transcranial photobiomodulation (PBM)
Implantable bioelectric stimulation:
   Central implants including
      Deep brain stimulation (DBS),
      Epidural cortical stimulation,
      Cranial cortical implants
      Spinal cord implants
   Peripheral implants including:
      Vagus nerve stimulation (VNS),
      Dorsal root ganglion (DRG) stimulation,
      Hypoglossal nerve stimulation,
      Peripheral nerve stimulation (PNS),
      Subcutaneous nerve stimulation
   and Genetically-targeted neuromodulation including:
      Optogenetic neuromodulation

The terms “enhancing” or “accelerating” refer broadly to improvements in therapeutic efficacy including but not limited to: acceleration of the onset or progression of therapeutic benefit, increasing durability or persistence of therapeutic outcomes, enhancement in magnitude or strength of therapeutic response, broadening the spectrum or scope of beneficial effects, reduction in required dosage, duration, or frequency of treatments, lowering the incidence, frequency, severity, or risk of adverse effects or side effects, improving treatment adherence or patient compliance, improving functional outcomes, overall health, or quality of life, increasing consistency, predictability, or reliability of therapeutic outcomes, preventing relapse or recurrence of symptoms, disorders, or conditions, facilitating recovery, rehabilitation, or restoration of function, reducing burden or complexity of treatment administration, and improving patient satisfaction, acceptance, or subjective experience of treatment.

The phrase “Individual therapeutic threshold” refers to the neuromodulation treatment threshold determined by a thresholding protocol appropriate to the neuromodulation method used, including but not limited to resting motor threshold for Transcranial Magnetic Stimulation (TMS) therapy; seizure threshold for Electroconvulsive Therapy (ECT) or Magnetic Seizure Therapy (MST); cardiovascular effects for Vagus Nerve Stimulation (VNS) or neurocardiac TMS; modulation of motor threshold or cardiovascular effects for Transcranial Direct Current Stimulation (TDCS) therapy, Transcranial Electrical Stimulation (LES), or transcranial focused ultrasound (tFUS) or electroencephalographic (EEG) or functional Magnetic Resonance Imaging (MRI) markers of neural response to stimulation (relevant to TMS, ECT, MST, VNS, TDCS, TES, tFUS, Deep Brain Stimulation (DBS), Epidural Cortical Stimulation).

As used herein the term plasticity-potentiating pharmaceutic agents includes,
N-methyl-D-aspartate (NMDA) receptor modulators such as
   NMDA receptor agonists, partial agonists (e.g., D-cycloserine, rapastinel, apimostinel)
   Glycine-site modulators (e.g., glycine, D-serine, sarcosine)
   Polyamine-site modulators (e.g., ifenprodil, eliprodil)
Non-NMDA glutamatergic modulators such as
   a-amino-3-hydroxy-5-methyl-4-isoxazolepropionic acid (AMPA)/kainate receptor modulators (e.g., aniracetam, CX-516, topiramate)
   Metabotropic glutamate receptor modulators (e.g., mavoglurant, LY341495)
   Glutamate release/reuptake modulators (e.g., lamotrigine, riluzole, ceftriaxone)
Monoaminergic modulators
Dopaminergic agents (e.g., levodopa, pramipexole, bupropion, lisdexamfetamine)
   Serotonergic agents (e.g., SSRIs, SNRIs, serotonin receptor ⋅ modulators)
   Adrenergic agents (e.g., propranolol, guanfacine)
   Histaminergic agents (e.g., betahistine)
Cholinergic modulators
   Acetylcholine receptor modulators (e.g., nicotine, varenicline, scopolamine)
   Cholinesterase inhibitors (e.g., donepezil, galantamine, rivastigmine)
GABAergic modulators
   gamma-aminobutyric acid (GABA) receptor modulators (e.g., benzodiazepines, baclofen)
   GABA reuptake/metabolism inhibitors (e.g., tiagabine, vigabatrin)
Opioidergic and endocannabinoid modulators
   Opioid receptor modulators (e.g., buprenorphine, naltrexone)
   Cannabinoid receptor modulators (e.g., cannabidiol, dronabinol)
   Endocannabinoid metabolism modulators (e.g., Fatty Acid Amide Hydrolase (FAAH) inhibitors)
Cellular signaling modulators
   Calcium signaling modulators (e.g., nimodipine, gabapentin, dantrolene, 1,2 BIS (2-aminophenoxy) ethene-N,N,N′,N′-tetraocetic acid tetrakis/acetoxymethyl ester (BAPTA-AM))
   Protein kinases/phosphatases (e.g., sirolimus/rapamycin, lithium)
   Secondary messenger modulators (e.g., forskolin, caffeine, Phosphodiesterase (PDE) inhibitors, GLP-1 receptor agonists such as semaglutide or tirzepatide)
Epigenetic and transcriptional modulators
   Histone and DNA methylation modulators (e.g., vorinostat, valproate, decitabine)
   Bromodomain modulators (e.g., JQ1)
   Transcription factor modulators (e.g., cAMP-responsive element modulator (CREB modulators, Nuclear factor kappa-light-chain β cell (NF-κB) modulators)
Structural and synaptic modulators
   Synaptic vesicle modulators (e.g., levetiracetam, brivaracetam)
   Cytoskeletal modulators (e.g., cytochalasin D, latrunculin A, paclitaxel)
   Dendritic spine modulators (e.g., Brain-derived neurotrophic factor (BDNF) modulators, ketamine, tianeptine)
   Synaptogenesis modulators (e.g., lithium, estrogen, insulin like growth (IGF-1))
   Axonal sprouting/pruning modulators (e.g., nerve growth factor (NGF), corticosteroids, semaphorins)
   Synaptic structural protein modulators (e.g., postsynaptic density protein-95 (PSD-95) modulators)
Neurotrophic and neuroimmune modulators
   Neurotrophic agents (e.g., BDNF, NGF, Glial cell line-derived neurotropic factor (GDNF), Tropomyosic receptor kinase B (TrkB) modulators)
   Immune and inflammatory modulators (e.g., minocycline, cytokine modulators)
Glial modulators
   Astrocyte modulators (e.g., fluoxetine, ibudilast)
   Microglia modulators (e.g., minocycline, resveratrol, low-dose naltrexone)
   Oligodendrocyte modulators (e.g., clemastine, biotin, sodium phenylbutyrate)

Targeted neurostimulation treatments of the brain such as TMS involve trains of stimuli to drive activity dependent changes in the brain, referred to as “synaptic plasticity”. The present disclosure is based on a discovery that administering certain drugs and drug combinations to a patient prior to, simultaneously with or following commencement of delivery of targeted neurostimulation treatments to the brain of a patient will significantly improve responsiveness of a patient's brain to a neurostimulation treatment by modulating or enhancing neuroplasticity of the brain.

In accordance with the present disclosure, we have found that various drug and drug combinations taken prior to, sequentially with or following commencement of external brain stimulation advantageously will significantly improve responsiveness of a patient's brain to neurostimulation treatment by modulating or enhancing neuroplasticity to the brain. The external brain stimulation should be delivered to the patient during a time period the drug or drug combination remains pharmacologically active. As a result, we are able to complete a full course of external brain stimulation treatment of a patient in need of same, including rest periods is significantly reduced, e.g., to a single work day or 8 hours, or less, depending on the underlying condition being treated and the particular brain stimulation system being employed.

More particularly the present disclosure provides a method for enhancing and/or accelerating the therapeutic effect of a neurostimulation treatment of the brain of a patient in need of such treatment, wherein said neuromodulation treatment comprises multiple sessions delivered over a period of time, comprising administering to the patient prior to, concurrently with, or following commencement of said administration of said neuromodulation treatment, a neuroplasticity-potentiating pharmaceutical agent, whereupon a total time of neuromodulation needed to achieve a desired therapeutic effect, including rest periods is significantly reduced e.g., to a single work day of 10 hours or less.

In one aspect the neuroplasticity-potentiating agent comprises a dopaminergic pharmacological agent.

In another aspect the dopaminergic pharmacological agent is a dopamine precursor agent, preferably L-DOPA (Levodopa).

In another aspect the dopaminergic pharmacological agent is a dopamine agonist, preferably Pramipexole, Ropinirole, Bromocriptine, Quinpirole, Rotigotine, or Apomorphine.

In yet another aspect the dopaminergic pharmacological agent is a monoamine oxidase inhibitor (MAOI), preferably Selegiline, Rasagiline, Phenelzine, or Tranylcypromine.

In a further aspect the dopaminergic pharmacological agent is a catechol-O-methyltransferase (COMT) inhibitor, preferably Entacapone, Tolcapone, or Opicapone.

In another aspect the dopaminergic pharmacological agent is a dopamine reuptake inhibitor (DRI), preferably Bupropion, Nomifensine, Amineptine, or Tianeptine.

In a further aspect the dopaminergic pharmacological agent is a combined dopaminergic therapy, preferably Sinemet (Levodopa/Carbidopa) or Stalevo (Levodopa/Carbidopa/Entacapone).

In yet another aspect the dopaminergic pharmacological agent is a stimulant increasing dopamine levels, preferably Amphetamine, Dextroamphetamine, Mixtures of Dextroamphetamine and Levoamphetamine, Lisdexamfetamine, Methamphetamine, Methylphenidate, or Dexmethylphenidate.

In another aspect the dopaminergic pharmacological agent is an adenosine receptor blocker, preferably Caffeine, Theophylline, Theobromine, Istradefylline, or Preladenant.

In another aspect the neurostimulation sessions are administered during a time period when said dopaminergic pharmaceutical agent remains pharmacologically active.

In another aspect the neuroplasticity-potentiating agent comprises a non-NMDA glutamatergic pharmacological agent.

In another aspect the glutamatergic pharmacological agent is an AMPA receptor modulator, preferably CX-516, CX-614, Aniracetam, Sunifiram, Piracetam, Pramiracetam, or Perampanel.

In yet another aspect the glutamatergic pharmacological agent is an AMPA receptor agonist, preferably Ibotenic Acid or Quisqualic Acid.

In a further aspect the glutamatergic pharmacological agent is a kainate receptor modulator, preferably Topiramate, LY466195, or NS102.

In an alternative aspect the glutamatergic pharmacological agent is a metabotropic glutamate receptor (mGluR) modulator, preferably LY341495, Mavoglurant, LY354740, or MPEP.

In another aspect the glutamatergic pharmacological agent is a glutamate release modulator, preferably Lamotrigine or Riluzole.

In a further aspect the glutamatergic pharmacological agent is a glutamate reuptake modulator, preferably Ceftriaxone.

In another aspect the glutamatergic pharmacological agent is a vesicular glutamate transporter (VGLUT) modulator, preferably Furosemide.

In yet another aspect the neurostimulation sessions are administered during a time period when said non-NMDA glutamatergic pharmacological agents remains pharmacologically active.

In a further aspect the neuroplasticity-potentiating agent comprises a dopaminergic pharmacological agent and an NMDAergic pharmacological agent.

In a further aspect the NMDA receptor pharmacological agent is an NMDA receptor agonist or partial agonist, preferably Cycloserine, D-Cycloserine, Rapastinel, Apimostinel, or Zelquistinel.

In yet another aspect the NMDA receptor pharmacological agent is a glycine site modulator (NR1 co-agonist), preferably Glycine, D-Serine, D-Alanine, or Sarcosine.

In another aspect the NMDA receptor pharmacological agent is a polyamine site modulator, preferably Ifenprodil or Eliprodil.

In yet another aspect the neuroplasticity-potentiating agent comprises a cholinergic agent, a calcium-mediated plasticity agent, a cytoskeletal restructuring agent, a chromatin remodeling agent, a synaptic reorganization agent or a neutropic agent.

In a further aspect the neurostimulation sessions are administered during a time period when said neuroplasticity-potentiating agent remains pharmacologically active.

In another aspect the neurostimulation treatment comprises transcranial magnetic stimulation (TMS).

In a further aspect the neurostimulation treatment comprises transcranial electrical stimulation of the brain via a non-convulsive technique such as transcranial direct current stimulation (DCS), transcranial alternating current stimulation (tACS), transcranial random noise stimulation, (RNS), transcranial temporal interference stimulation (tTIS), or deep brain stimulation (DBS), or minimally invasive bioelectric implantation.

In a still further aspect the neurostimulation treatment comprises a convulsive technique such as electroconvulsive therapy (ECT) or magnetic seizure therapy (MST).

In yet another aspect the targeted neurostimulation treatment comprises treatment with transcranial focused ultrasound (FUS) or transcranial photobiomodulation.

In a still further aspect the treatment is for major depressive disorder and/or anxiety.

In yet another aspect the treatment is for a psychiatric disorders preferably: bipolar disorder; obsessive-compulsive disorder; post-traumatic stress disorder; other anxiety disorders (including but not limited to generalized anxiety disorder, panic disorder, social anxiety disorder, or specific phobias such as fear of flying); attention deficit/hyperactivity disorder; Tourette syndrome, body-focused repetitive behaviors; substance abuse or dependence; sleep disorders including primary or secondary insomnia; eating disorders such as anorexia nervosa, bulimia nervosa, or binge eating disorder; impulse control disorders such as pathological gambling; psychotic disorders such as schizophrenia, schizophreniform disorder; autism spectrum disorders; or a personality disorder such as borderline personality disorder or obsessive-compulsive personality disorder.

In a further aspect the treatment is for a neurodegenerative disorder selected from the group consisting of Alzheimer's disease, Parkinson's disease, Huntington's disease, corticobasal degeneration, spinocerebellar ataxia, amyotrophic lateral sclerosis, frontotemporal dementia, chronic traumatic encephalopathy, and post-stroke neurodegeneration.

In another aspect the treatment is for a neurological disorder selected from the group consisting of tinnitus, migraine, stroke, epilepsy, multiple sclerosis, and chronic pain.

Various classes of exogenous drugs and methods that influence neuro-structural plasticity, neurotrophic factors, and glial cell activity may be used in the practice of the present disclosure. Structural plasticity involves dendritic spine remodeling, synaptogenesis, and axonal sprouting/pruning, with drugs such as Brain-derived Neurotropic Factor (BDNF), ketamine, and lithium modulating these processes. Neurotrophic factors such as BDNF, Nerve Growth Factor (NGF), and Global cell line-derived neurotropic factor (GDNF) promote neuronal survival and plasticity, with drugs such as SSRIs, melatonin, and dopamine agonists enhancing their expression. Glial cells, including astrocytes, microglia, and oligodendrocytes, support Central Neuron System (CNS) functions, and drugs like fluoxetine, minocycline, and clemastine regulate their activities, potentially improving neuroplasticity and repair.

The dopaminergic system involves modulating dopamine levels through a variety of drugs that impact synthesis, release, reuptake, and receptor activity. Exogenous compounds such as L-DOPA, dopamine agonists, and dopamine reuptake inhibitors may be used to enhance or reduce dopamine's effects, impacting neuroplasticity, mood, and motor function.

In a further aspect of the disclosure there is provided a method for enhancing and/or accelerating the therapeutic effects of psychedelic treatment of the brain of an individual, comprising administering to said patient prior to, concurrently with, or following administration of said psychedelic treatment one or more neuroplasticity-potentiating pharmacological agents, wherein said psychedelic treatment includes the administration of one or more psychedelic compounds.

In a preferred embodiment the psychedelic compound is selected from LSD, psilocybin, MDMA, ibogaine, DMT, ayahuasca, ketamine, mescaline.

In still yet another aspect of the disclosure there is provided a method for enhancing and/or accelerating the therapeutic effects of psychotherapy for an individual, comprising administering to said patient prior to, concurrently with, or following psychotherapy treatment one or more neuroplasticity-potentiating pharmacological agents, wherein said psychedelic treatment includes the administration of one or more psychedelic compounds. whereupon the neuroplasticity potentiating pharmaceutical agent comprises A) a dopaminergic pharmacological agent, a non-NMDA glutamatergic pharmacological agent, an NMDAergic pharmacological agent, a cholinergic agent, a calcium-mediated plasticity agent, a cytoskeletal restructuring agent, a chromatin remodeling agent, a synaptic reorganization agent or a neurotropic agent and optionally B) D-cycloserine (DCS), a N-methyl-D-aspartate receptor agonist (NMDA), or DCS and NMDA.

In one embodiment the said neuroplasticity-potentiating agent is selected from the group consisting of dopaminergic pharmacological agent, preferably a dopamine precursor agent, more preferably L-DOPA (Levodopa); a dopamine agonist, preferably Pramipexole, Ropinirole, Bromocriptine, Quinpirole, Rotigotine, or Apomorphine; a monoamine oxidase inhibitor (MAOI), preferably Selegiline, Rasagiline, Phenelzine, or Tranylcypromine; a catechol-O-methyltransferase (COMT) inhibitor, preferably Entacapone, Tolcapone, or Opicapone; a dopamine reuptake inhibitor (DRI), preferably Bupropion, Nomifensine, Amineptine, or Tianeptine; a combined dopaminergic therapy, preferably Sinemet (Levodopa/Carbidopa) or Stalevo (Levodopa/Carbidopa/Entacapone); a stimulant increasing dopamine levels, preferably Amphetamine, Dextroamphetamine, Mixtures of Dextroamphetamine and Levoamphetamine, Lisdexamfetamine, Methamphetamine, Methylphenidate, or Dexmethylphenidate; and an adenosine receptor blocker, preferably Caffeine, Theophylline, Theobromine, Istradefylline, or Preladenant.

Alternatively the neuroplasticity-potentiating agent comprises a non-NMDA glutamatergic pharmacological agent, selected from the group consisting of an AMPA receptor modulator, preferably CX-516, CX-614, Aniracetam, Sunifiram, Piracetam, Pramiracetam, or Perampanel; an AMPA receptor agonist, preferably Ibotenic Acid or Quisqualic Acid; a kainate receptor modulator, preferably Topiramate, LY466195, or NS102; a metabotropic glutamate receptor (mGluR) modulator, preferably LY341495, Mavoglurant, LY354740, or MPEP; a glutamate release modulator, preferably Lamotrigine or Riluzole; a glutamate reuptake modulator, preferably Ceftriaxone; and a vesicular glutamate transporter (VGLUT) modulator, preferably Furosemide.

As used herein the following definitions apply:

Dopaminergic Pharmacological Agent: A chemical agent that modulates the dopamine system in the brain. This can include agents that increase dopamine availability, stimulate dopamine receptors, inhibit dopamine reuptake, or prolong dopamine activity by inhibiting enzymes such as monoamine oxidase or catechol-O-methyltransferase.

Non-NMDA Glutamatergic Pharmacological Agent: A compound that modulates the glutamate system, excluding those that act on NMDA (N-Methyl-D-aspartate) receptors. These agents may include modulators or agonists of AMPA or kainate receptors, or other glutamate-related mechanisms.

NMDAergic Pharmacological Agent: A chemical that modulates NMDA receptors, which are a subtype of glutamate receptors involved in synaptic plasticity and memory function. These agents may act as NMDA receptor agonists, partial agonists, or modulators of specific sites on the NMDA receptor, such as the glycine or polyamine binding sites.

Neuroplasticity-potentiating pharmacological agents refer to a class of compounds that modulate neuroplasticity by enhancing or accelerating the brain's ability to reorganize itself, form new neural connections, or strengthen existing ones in response to various forms of neuromodulation or treatment. These agents can act via multiple mechanisms, such as:
1. Modulating neurotransmitter systems (e.g., dopaminergic, glutamatergic) by influencing receptor activity, reuptake, or degradation.
2. Affecting intracellular processes like cytoskeletal restructuring or chromatin remodeling to facilitate synaptic reorganization and promote neuroplasticity.
3. Supporting synaptic plasticity by interacting with neurotrophic factors or signaling pathways critical for neural growth and repair.

These agents encompass various mechanisms, including but not limited to agonists, antagonists, reuptake inhibitors, enzyme inhibitors, and other modulators of neurotransmitter systems. In the context of your claims, these pharmacological agents are applied in conjunction with device-based neurostimulation or alternative treatments (e.g., psychedelic compounds, psychotherapy) to enhance the therapeutic effects, either by potentiating synaptic efficacy, promoting neural circuit reorganization, or facilitating faster recovery from neurological conditions.

Pharmacologically Active Period: The duration during which a pharmacological agent remains sufficiently active in the body to exert its intended therapeutic effect. This period is defined as 5 half-lives of the agent, at which point approximately 3.13% of the original dose remains active.

A course of neurostimulation: A treatment regimen that consists of one or more sessions of neuromodulation. Each session typically lasts from a few seconds to about an hour, with common durations being approximately 3 minutes, 10 minutes, and 38 minutes. During sessions, neuromodulation is often—but not always—delivered in bursts or trains of pulses, which may be interrupted by brief pauses, usually lasting between 8 and 30 seconds. Any pause exceeding one minute is generally considered to mark the end of one session, and any pulses delivered after such a pause would be counted as part of a new session. It is conceivable that neuromodulation may also be effectively delivered in one very long session—for example, pulses at 3 Hz with short or no breaks for multiple hours.

Discussed below are various drugs that are useful in the practice of the instant disclosure:

A) Various Useful Exogenous Drugs that Influence Dopamine Levels Including:

### Stimulants Such as:

Amphetamines (e.g., Adderall, Dextroamphetamine) which act to boost dopamine by reversing the dopamine transporter (DAT) and increasing release from vesicles. Used for ADHD and narcolepsy.
Methamphetamine which act to increase dopamine release and blocks reuptake, leading to euphoria.
Methylphenidate (Ritalin) which act to inhibit DAT, raising dopamine in the synapse. Used for ADHD.
Cocaine which acts to block DAT, preventing reuptake and enhancing dopamine levels, causing euphoria.

### Dopamine Precursors Such as:

L-DOPA (Levodopa) which is a precursor that converts to dopamine, used in Parkinson's disease.
Carbidopa which acts to inhibit peripheral conversion of L-DOPA to dopamine, ensuring more reaches the brain. Often combined with L-DOPA (e.g., Sinemet).

### Dopamine Agonists Such as:

Pramipexole which acts to stimulate dopamine receptors directly, used in Parkinson's and restless legs syndrome.
Ropinirole which is another dopamine agonist for Parkinson's and restless legs syndrome.
Bromocriptine which is a dopamine agonist that also reduces prolactin, used for Parkinson's and hyperprolactinemia.
Quinpirole which is a dopamine autoreceptor agonist that reduces dopamine release, primarily used in research.

### Dopamine Antagonists Such as:

Haloperidol which acts to block dopamine receptors, used as an antipsychotic.
Sulpiride which acts to block autoreceptors, increasing dopamine release, used as an antipsychotic and antidepressant.

### Monoamine Oxidase Inhibitors (MAOIs) Such as:

Selegiline which acts to inhibit MAO-B, preventing dopamine breakdown. Used in Parkinson's and depression.
Rasagiline which is similar to Selegiline, used in Parkinson's to increase dopamine.
Phenelzine which is a non-selective MAOI, increasing dopamine, norepinephrine, and serotonin, used in depression.

### Catechol-O-Methyltransferase (COMT) Inhibitors Such as:

Entacapone which acts to inhibit COMT, prolonging L-DOPA's effects in Parkinson's.
Tolcapone which is another COMT inhibitor that extends L-DOPA's duration.

### Vesicular Monoamine Transporter (VMAT2) Inhibitors Such as:

Reserpine which acts to deplete dopamine storage by inhibiting VMAT2, historically used for hypertension.
Tetrabenazine which acts to reduce dopamine levels, used in hyperkinetic movement disorders like Huntington's.

### Dopamine Reuptake Inhibitors (DRIs) Such as:

Bupropion which acts to inhibits reuptake of dopamine and norepinephrine, used as an antidepressant and for smoking cessation.

### Dopamine Releasers Such as:

Amphetamine which acts to increase dopamine release and inhibits reuptake, used in ADHD and narcolepsy.
Dextroamphetamine which is a more potent isomer of amphetamine, used similarly for ADHD and narcolepsy.

### Combined Dopamine Therapy Such as:

Sinemet (Levodopa/Carbidopa) which acts to increase brain dopamine while reducing peripheral side effects of L-DOPA.

### Methods of Modifying Synaptic Dopamine Levels

Below are discussions of various methods for modifying synaptic dopamine levels, including processes, enzymes or receptors involved, and examples of exogenous drugs that affect these processes:

### Dopamine Synthesis

Dopamine synthesis occurs in neurons through the conversion of the amino acid tyrosine to dopamine.

### Processes

Tyrosine Hydroxylation (TH) which acts to convert tyrosine into L-DOPA.
Decarboxylation: Aromatic L-amino acid decarboxylase (AADC) which acts to convert L-DOPA into dopamine.

### Enzymes

Tyrosine Hydroxylase (TH) which is a rate-limiting enzyme in dopamine synthesis.
Aromatic L-amino acid decarboxylase (AADC) which acts to convert L-DOPA into dopamine.

### Exogenous Drugs

L-DOPA (Levodopa) which acts as a precursor to dopamine, used to increase dopamine synthesis in the treatment of Parkinson's disease.
Carbidopa which acts to inhibit AADC outside the brain, increasing L-DOPA availability for conversion to dopamine within the brain.

### Dopamine Release

Dopamine release into the synaptic cleft is triggered by the arrival of an action potential at the presynaptic terminal.

### Process

Vesicular Release: Dopamine is stored in vesicles within presynaptic neurons and released into the synapse upon neuronal firing.
Calcium-Dependent Exocytosis: Dopamine release is calcium-dependent and involves the fusion of vesicles with the presynaptic membrane.

### Enzymes/Receptors

Vesicular Monoamine Transporter (VMAT2): Transports dopamine into vesicles for storage.
SNARE Proteins: Mediate the fusion of vesicles with the presynaptic membrane.

### Exogenous Drugs

Amphetamines: e.g., Adderall—which acts to increase dopamine release by reversing the dopamine transporter (DAT) and promoting dopamine release from vesicles.
Methamphetamine which acts to increase dopamine release and blocks dopamine reuptake.

### Dopamine Reuptake

Dopamine reuptake involves the removal of dopamine from the synaptic cleft back into the presynaptic neuron, effectively terminating its action.

### Process

Dopamine Transporter (DAT): The primary mechanism by which dopamine is removed from the synaptic cleft and returned to the presynaptic neuron.

### Enzymes/Receptors

Dopamine Transporter (DAT): Responsible for reuptake of dopamine from the synaptic cleft.

### Exogenous Drugs

Cocaine which acts to block DAT, preventing dopamine reuptake and increasing dopamine levels in the synapse.
Methylphenidate (Ritalin) which acts to inhibit DAT, increasing dopamine levels in the synapse.

### Dopamine Degradation

Dopamine degradation involves the breakdown of dopamine by specific enzymes, reducing dopamine levels in the brain.

### Process

Monoamine Oxidase (MAO) which acts to degrade dopamine into inactive metabolites.
Catechol-O-Methyltransferase (COMT) which acts to methylate dopamine, leading to its inactivation.

### Enzymes

Monoamine Oxidase (MAO-A and MAO-B) which acts to break down dopamine into DOPAC.
Catechol-O-Methyltransferase (COMT) which acts to convert dopamine into 3-methoxytyramine.

### Exogenous Drugs

MAO Inhibitors (MAOIs): e.g., Selegiline (MAO-B inhibitor)—which acts to prevent dopamine breakdown, increasing dopamine levels.
COMT Inhibitors: e.g., Entacapone—which acts to inhibit COMT, used to prolong the effect of L-DOPA in Parkinson's Disease treatment.

### Dopamine Receptor Modulation

Dopamine receptors, particularly D1-like (D1, D5) and D2-like (D2, D3, D4) receptors, mediate the effects of dopamine in the brain.

### Process

Receptor Activation/Inhibition: Dopamine binds to its receptors, modulating neuronal activity.

### Enzymes/Receptors

D1-like Receptors (D1, D5) which acts to stimulate adenylyl cyclase and increase cAMP levels, leading to excitatory effects.
D2-like Receptors (D2, D3, D4) which acts to inhibit adenylyl cyclase, decreasing CAMP levels and leading to inhibitory effects.

### Exogenous Drugs

Dopamine Agonists: e.g., Pramipexole (D2/D3 agonist)—which is used to treat Parkinson's Disease by directly stimulating dopamine receptors.
Dopamine Antagonists: e.g., Haloperidol (D2 antagonist)—which is used as an antipsychotic by blocking dopamine receptors.

### Presynaptic Dopamine Autoreceptor Modulation

Presynaptic dopamine autoreceptors regulate dopamine release by providing negative feedback to the neuron.

### Process

Autoreceptor Activation: When activated by dopamine, these receptors reduce further dopamine release, acting as a feedback mechanism.

### Enzymes/Receptors

D2 Autoreceptors: Located on the presynaptic neuron, these receptors inhibit dopamine release when activated.

### Exogenous Drugs

Dopamine Autoreceptor Agonists: e.g., Quinpirole—which acts to selectively activate D2 autoreceptors, reducing dopamine release.
Dopamine Autoreceptor Antagonists: e.g., Sulpiride—which acts to block D2 autoreceptors, increasing dopamine release.

### Dopamine Storage and Vesicular Transport

Dopamine storage within vesicles is crucial for its regulated release into the synapse.

### Process

Vesicular Storage: Dopamine is transported into vesicles by VMAT2 for storage until release.

### Enzymes/Receptors

Vesicular Monoamine Transporter (VMAT2): Transports dopamine into synaptic vesicles.

### Exogenous Drugs

Reserpine which acts to inhibit VMAT2, leading to depletion of dopamine storage and reduced synaptic dopamine levels.
Tetrabenazine which acts to inhibit VMAT2, and used to decrease dopamine levels, particularly in the treatment of hyperkinetic movement disorders.

### Dopamine Transport Across the Blood-Brain Barrier

Transport of dopamine precursors or analogs across the blood-brain barrier (BBB) can influence dopamine levels in the brain.

### Process

L-DOPA Transport: L-DOPA crosses the BBB and is converted to dopamine in the brain, while dopamine itself does not easily cross the BBB.

### Enzymes/Receptors

Amino Acid Transporters: Transport L-DOPA across the BBB.

### Exogenous Drugs

Levodopa (L-DOPA): Crosses the BBB and is converted to dopamine in the brain.
Carbidopa is co-administered with L-DOPA to inhibit peripheral AADC and enhance L-DOPA's brain availability.
   B) Exogenous Drugs that Influence the Cholinergic System

### Cholinergic Agonists Such as:

Acetylcholine which acts as a primary neurotransmitter and typically is not used as a drug due to rapid breakdown.
Pilocarpine which acts as a muscarinic agonist for dry mouth and glaucoma.
Bethanechol which acts as a muscarinic agonist for urinary retention.
Cevimeline which acts as a muscarinic agonist for dry mouth in Sjögren's syndrome.

### Cholinesterase Inhibitors Such as:

Donepezil which acts to inhibit acetylcholinesterase, used for treatment of Alzheimer's Disease.
Rivastigmine which acts as a reversible cholinesterase inhibitor used for treatment of for Alzheimer's Disease and Parkinson's Disease.
Galantamine which acts to inhibit acetylcholinesterase and modulates nicotinic receptors, used for treatment of Alzheimer's Disease.
Neostigmine which is a peripheral cholinesterase inhibitor used for treatment for myasthenia gravis and for reversing muscle relaxants.
Pyridostigmine which is similar to neostigmine, and is used for treatment of myasthenia gravis.

### Nicotinic Receptor Agonists Such as:

Nicotine which activates nicotinic receptors, used in smoking cessation.
Varenicline (Chantix) which is a partial agonist at nicotinic receptors, used for smoking cessation.
Cytisine which is a partial agonist at nicotinic receptors, used for smoking cessation.

### Nicotinic Receptor Antagonists Such as:

Mecamylamine which is a non-selective nicotinic antagonist, used as an antihypertensive.
Bupropion which is a dopamine reuptake inhibitor and nicotinic antagonist, used in smoking cessation.

### Muscarinic Receptor Antagonists Such as:

Atropine which is a non-selective muscarinic antagonist, used for treating bradycardia and as an antidote for poisoning.
Scopolamine which is a muscarinic antagonist used for treating motion sickness.
Ipratropium which is a muscarinic antagonist, used as a bronchodilator for treating Chronic Obstruction Pulmonary Disease (COPD).
Oxybutynin which is a muscarinic antagonist used for treating overactive bladder.
Benztropine which is used for treating Parkinson's Disease and drug-induced symptoms by blocking central muscarinic receptors.

### Choline Transport Inhibitors Such as:

Hemicholinium-3 which acts to inhibit choline transporter, reducing acetylcholine synthesis; used in research.

### Acetylcholine Release Modulators Such as:

Botulinum Toxin (Botox) which acts to inhibit acetylcholine release, reducing muscle activity; used in medical and cosmetic treatments.
α-Latrotoxin which is a toxin that triggers massive acetylcholine release; used in research.

### Acetylcholine Receptor Modulators Such as:

Galantamine which acts as a positive modulator of nicotinic receptors, enhancing cholinergic transmission.

### Methods of Modifying the Cholinergic System

### Acetylcholine Synthesis

Acetylcholine synthesis occurs in cholinergic neurons, primarily in the basal forebrain, brainstem, and neuromuscular junctions.

### Process

Choline Uptake: Choline is taken up by the neuron via a high-affinity transporter.
Acetylcholine Synthesis: Choline acetyltransferase (ChAT) catalyzes the transfer of an acetyl group from acetyl-CoA to choline, producing acetylcholine.

### Enzymes

Choline Acetyltransferase (ChAT): The enzyme responsible for synthesizing acetylcholine from choline and acetyl-CoA.

### Exogenous Drugs

Hemicholinium-3 which acts to inhibit choline uptake, reducing acetylcholine synthesis.
Lecithin which is a dietary source of choline that can increase acetylcholine synthesis when taken as a supplement.

### Acetylcholine Release

Acetylcholine release occurs when an action potential triggers the fusion of vesicles with the presynaptic membrane.

### Process

Vesicular Storage: Acetylcholine is stored in vesicles within the presynaptic neuron.
Calcium-Dependent Exocytosis: Upon depolarization, calcium influx triggers the fusion of vesicles with the presynaptic membrane, releasing acetylcholine into the synaptic cleft.

### Enzymes/Receptors

Vesicular Acetylcholine Transporter (VAChT): Transports acetylcholine into vesicles for storage.
SNARE Proteins: Mediate the fusion of vesicles with the presynaptic membrane.

### Exogenous Drugs

Vesamicol which acts to inhibit VAChT, reducing acetylcholine storage and release.
Botulinum Toxin which acts to inhibit acetylcholine release by cleaving SNARE proteins necessary for vesicle fusion.

### Acetylcholine Degradation

Acetylcholine degradation is the primary mechanism by which cholinergic signaling is terminated in the synaptic cleft.

### Process

Hydrolysis: Acetylcholine is rapidly broken down into choline and acetate by acetylcholinesterase.

### Enzymes

Acetylcholinesterase (AChE): The enzyme responsible for degrading acetylcholine in the synaptic cleft.

### Exogenous Drugs

Acetylcholinesterase Inhibitors: e.g., Donepezil, Rivastigmine and Galantamine-which act to inhibit AChE, increasing acetylcholine levels in the synapse.

### Cholinergic Receptor Modulation

Cholinergic receptors are of two main types: nicotinic receptors, which are ion channels, and muscarinic receptors, which are G-protein-coupled receptors.

### Nicotinic Receptors

Process: Activation by acetylcholine or agonists leads to ion channel opening and depolarization of the postsynaptic neuron.
Receptors: Nicotinic acetylcholine receptors (nAChRs) are found in the autonomic ganglia, neuromuscular junctions, and the central nervous system.

### Muscarinic Receptors

Process: Activation by acetylcholine or agonists leads to various intracellular signaling cascades via G-proteins.
Receptors: Muscarinic acetylcholine receptors (mAChRs) are found throughout the central and peripheral nervous systems.

### Exogenous Drugs

Cholinergic Agonists: e.g., Bethanechol, Pilocarpine (muscarinic agonists); and Nicotine (nicotinic agonist)—which act to stimulate cholinergic receptors.
Cholinergic Antagonists: e.g., Atropine, Scopolamine (muscarinic antagonists); and Mecamylamine (nicotinic antagonist)—which act to inhibit cholinergic receptors.

### Choline Uptake and Storage

Choline uptake into presynaptic neurons is crucial for acetylcholine synthesis and storage.

### Process

Choline Uptake: Choline is transported into the presynaptic neuron by a high-affinity choline transporter (CHT).
Vesicular Storage: Acetylcholine is stored in synaptic vesicles via VAChT.

### Enzymes/Receptors

Choline Transporter (CHT): Responsible for the uptake of choline into the neuron.
Vesicular Acetylcholine Transporter (VAChT): Responsible for transporting acetylcholine into synaptic vesicles.

### Exogenous Drugs

Hemicholinium-3 which acts to inhibit choline uptake into presynaptic neurons, reducing acetylcholine synthesis.
Vesamicol which acts to inhibit VAChT, reducing acetylcholine storage and release.
   C) Exogenous Drugs that Influence the Glutamatergic System (Excluding NMDA) AMPA Receptor Modulators Such as:
AMPAkines (e.g., CX-516) which act as positive allosteric modulators that enhance glutamatergic transmission.
Perampanel which acts as a non-competitive antagonist used to treat epilepsy by reducing excitatory neurotransmission.
Aniracetam which acts as a positive modulator used as a cognitive enhancer.

### Kainate Receptor Modulators Such as:

Topiramate which acts as an antagonist at kainate receptors, used as an anticonvulsant and for migraine prevention.
LY466195 which acts as a selective kainate receptor antagonist with potential neuroprotective effects.
NS102 which acts as a kainate receptor antagonist used in research.
   mGluR (Metabotropic Glutamate Receptor) Modulators Such as:
LY341495 which acts as a selective mGluR2/3 antagonist that modulates glutamatergic neurotransmission.
Mavoglurant which acts as an mGluR5 antagonist being investigated for fragile X syndrome and other conditions.
LY354740 which acts as a Group II mGluR agonist that reduces glutamate release, potential anxiolytic.
MPEP which acts as an mGluR5 antagonist used in research to reduce excitotoxicity and anxiety.

### Glutamate Release Modulators Such as

Lamotrigine which acts to inhibit voltage-gated sodium channels, reducing glutamate release; used as an anticonvulsant.
Riluzole which acts to decrease glutamate release, used to treat ALS.
Gabapentin which acts to inhibit glutamate release by blocking calcium channels, used for neuropathic pain and epilepsy.

### Glutamate Reuptake Modulators Such as:

Ceftriaxone which acts to enhance EAAT2 expression, increasing glutamate reuptake and reducing extracellular levels.
Tianeptine which acts to enhance glutamate reuptake, though primarily modulates serotonin.

### Vesicular Glutamate Transporter (VGLUT) Modulators Such as:

Rose Bengal which acts to inhibit VGLUT, reducing glutamate storage in synaptic vesicles; used in research.

### Methods of Modifying the Glutamatergic System (Excluding NMDA Receptor Activation) Glutamate Synthesis

Glutamate is synthesized in neurons and glial cells and is the primary excitatory neurotransmitter in the central nervous system.

### Process

Glutamine Conversion: Glutamine is converted into glutamate by the enzyme glutaminase in neurons.

### Enzymes

Glutaminase is used to convert glutamine into glutamate within the neuron.

### Exogenous Drugs

6-Diazo-5-oxo-L-norleucine (DON) which acts to inhibit glutaminase, reducing the synthesis of glutamate.

### Glutamate Release

Glutamate release into the synaptic cleft occurs upon depolarization of the presynaptic neuron.

### Process

Vesicular Storage: Glutamate is stored in synaptic vesicles via vesicular glutamate transporters (VGLUTs).
Calcium-Dependent Exocytosis: Depolarization causes calcium influx, triggering the fusion of glutamate-containing vesicles with the presynaptic membrane.

### Enzymes/Receptors

Vesicular Glutamate Transporters (VGLUTs): Transport glutamate into synaptic vesicles for storage.
SNARE Proteins: Mediate the fusion of vesicles with the presynaptic membrane.

### Exogenous Drugs

Lamotrigine which acts to inhibit voltage-gated sodium channels, reducing the release of glutamate.
Riluzole which acts to decrease glutamate release by blocking voltage-gated sodium channels and inhibiting glutamate release.

### Glutamate Reuptake

Glutamate reuptake is crucial for terminating synaptic transmission and preventing excitotoxicity.

### Process

Reuptake by Transporters: Glutamate is taken up by excitatory amino acid transporters (EAATs) on neurons and glial cells.

### Enzymes/Receptors

Excitatory Amino Acid Transporters (EAATs): Responsible for the reuptake of glutamate from the synaptic cleft.

### Exogenous Drugs

Ceftriaxone which acts to increase EAAT2 expression, enhancing glutamate reuptake.
Tianeptine which acts to enhance glutamate reuptake, modulating synaptic glutamate levels.

### Ampakine (AMPA) Receptor Modulation

AMPA receptors mediate fast excitatory synaptic transmission in the central nervous system.

### Process

Receptor Activation: Glutamate binds to AMPA receptors, causing the opening of ion channels and depolarization of the postsynaptic neuron.

### Receptors

AMPA Receptors: Ionotropic receptors that mediate fast excitatory neurotransmission.

### Exogenous Drugs

AMPAkines: e.g., CX-516-positive allosteric modulators that enhance AMPA receptor activity and glutamatergic transmission.
Perampanel which acts as an AMPA receptor antagonist that reduces excitatory neurotransmission, used in epilepsy treatment.

### Kainate Receptor Modulation

Kainate receptors are another class of ionotropic glutamate receptors involved in excitatory neurotransmission.

### Process

Receptor Activation: Glutamate binding to kainate receptors causes ion channel opening, leading to depolarization of the postsynaptic neuron.

### Receptors

Kainate Receptors: Ionotropic receptors that contribute to excitatory neurotransmission and are involved in synaptic plasticity.

### Exogenous Drugs

Topiramate which acts as a kainate receptor antagonist used as an anticonvulsant and in migraine prevention.
LY466195 which acts as a selective kainate receptor antagonist with potential neuroprotective effects.
   mGluR Modulation (Metabotropic Glutamate Receptors)

Metabotropic glutamate receptors (mGluRs) are G-protein-coupled receptors that modulate glutamatergic transmission and synaptic plasticity.

### Process

Receptor Activation: mGluRs modulate intracellular signaling cascades that can influence neurotransmitter release, synaptic plasticity, and neuronal excitability.

### Receptors

mGluR1-8: Different subtypes of metabotropic glutamate receptors, which are categorized into three groups based on their sequence homology, signal transduction pathways, and pharmacological profiles.

### Exogenous Drugs

LY341495 which acts as a selective mGluR2/3 antagonist that can modulate glutamatergic neurotransmission.
Mavoglurant which acts as an mGluR5 antagonist being investigated for its potential to treat fragile X syndrome and other conditions.
   D) Exogenous Drugs that Influence NMDA Receptor Activity

### NMDA Receptor Antagonists Such as:

Ketamine which acts as a non-competitive NMDA receptor antagonist, used for its rapid antidepressant effects and as an anesthetic.
Memantine which acts as an NMDA receptor antagonist used in the treatment of Alzheimer's disease to prevent excitotoxicity.
Amantadine which acts as an NMDA receptor antagonist used in Parkinson's disease and for the treatment of drug-induced dyskinesias.

### Glycine Site Modulators Such as:

D-cycloserine which acts as a partial agonist at the glycine site of the NMDA receptor, used to enhance cognitive function and in the treatment of anxiety disorders.
Sarcosine which acts as a glycine transporter inhibitor that increases glycine levels, enhancing NMDA receptor function and showing potential in treating schizophrenia.

### Polyamine Site Modulators Such as:

Ifenprodil which acts as a selective antagonist of the NR2B subunit of the NMDA receptor, used in research for its neuroprotective properties.
Eliprodil which acts as another NR2B-selective antagonist, investigated for its potential neuroprotective effects in ischemic brain injury.

### Co-Agonists Such as:

Glycine which acts as an endogenous co-agonist required for NMDA receptor activation, enhancing receptor function when applied exogenously.
D-Serine which acts as another co-agonist of the NMDA receptor, often used to enhance NMDA receptor-mediated neurotransmission.

### NMDA Receptor Overview

The NMDA receptor is a type of ionotropic glutamate receptor that plays a critical role in synaptic plasticity, learning, and memory. It is unique among glutamate receptors due to its voltage-dependent activation, requiring both ligand binding and membrane depolarization.

### Structure

Subunits: The NMDA receptor is composed of four subunits, typically including two NR1 subunits and two NR2 or NR3 subunits. The combination of these subunits determines the receptor's properties and pharmacology.
Binding Sites: The receptor has multiple binding sites, including those for glutamate, glycine (a co-agonist), zinc, polyamines, and magnesium.

### Function

Calcium Permeability: Upon activation, the NMDA receptor allows calcium (Ca²⁺), sodium (Na⁺), and potassium (K⁺) ions to flow through the channel, leading to the activation of various intracellular signaling cascades.
Voltage-Dependent Block: The receptor is blocked by magnesium (Mg²⁺) at resting membrane potentials, which is relieved upon depolarization, allowing ion flow.

### Exogenous Drugs

Ketamine which acts to block NMDA receptors, providing rapid antidepressant effects and acting as an anesthetic.
Memantine which is a low-affinity antagonist that prevents excessive activation of NMDA receptors, protecting against excitotoxicity in neurodegenerative diseases.

### NMDA Receptor Modulation

### Glycine Site Modulation

The glycine site on the NMDA receptor is a co-agonist site that must be occupied by glycine or D-serine for the receptor to be activated by glutamate.

### Process

Co-Agonist Binding: Glycine or D-serine binds to the glycine site, allowing the NMDA receptor to respond to glutamate and initiate ion flow.

### Sites and Subunits

NR1 Subunit which contains the binding site for glycine, which is essential for receptor activation.
NR3 Subunit which modulates glycine binding and overall receptor function.

### Exogenous Drugs

D-cycloserine which acts as a partial agonist at the glycine site, enhancing NMDA receptor function in cognitive enhancement and anxiety treatment.
Sarcosine which acts to inhibit glycine reuptake, increasing glycine levels and enhancing NMDA receptor activity.

### Polyamine Site Modulation

Polyamines, such as spermine and spermidine, bind to specific sites on the NMDA receptor and can modulate its function, often enhancing receptor activity.

### Process

Polyamine Binding: Polyamines bind to allosteric sites on the NMDA receptor, which can potentiate or inhibit receptor activity depending on the receptor subunit composition.

### Sites and Subunits

NR2B Subunit: Particularly involved in polyamine modulation, with binding sites that can influence receptor activity.

### Exogenous Drugs.

Ifenprodil which acts as a selective antagonist of the NR2B subunit, reducing the potentiation of NMDA receptors by polyamines and offering neuroprotective effects.
Eliprodil which acts as another NR2B antagonist, with potential therapeutic applications in neuroprotection.

### Magnesium Block

The NMDA receptor is unique in its voltage-dependent block by magnesium ions, which is relieved upon depolarization of the postsynaptic membrane.

### Process

Magnesium Block: At resting membrane potentials, magnesium ions block the NMDA receptor channel, preventing ion flow.
Depolarization Relief: Upon depolarization, magnesium is expelled from the channel, allowing ions to pass through the receptor.

### Sites and Subunits

Pore Region: The channel pore is where magnesium ions block ion flow under resting conditions.

### Exogenous Drugs

Magnesium Supplements: Can modulate NMDA receptor activity by affecting the extent of the voltage-dependent block.

### Calcium Influx and Signal Transduction

One of the critical functions of the NMDA receptor is its permeability to calcium ions, which act as a secondary messenger in various intracellular signaling pathways.

### Process

Calcium Entry: Upon activation, the NMDA receptor allows calcium to enter the cell, triggering signaling pathways involved in synaptic plasticity, gene expression, and neuroprotection.

### Signaling Pathways

CaMKII Activation: Calcium influx activates calcium/calmodulin-dependent protein kinase II (CaMKII), which plays a critical role in long-term potentiation (LTP).
CREB Phosphorylation: Calcium also activates pathways that lead to the phosphorylation of CREB (CAMP response element-binding protein), a transcription factor involved in synaptic plasticity and memory formation.

### Exogenous Drugs

Calcium Channel Blockers (e.g., Verapamil): While not directly targeting NMDA receptors, these can influence calcium dynamics and potentially affect downstream signaling pathways linked to NMDA receptor activity.

### Excitotoxicity and Neuroprotection

Overactivation of NMDA receptors can lead to excitotoxicity, a process where excessive calcium influx triggers cell death. This is implicated in various neurodegenerative diseases.

### Process

Excitotoxicity: Prolonged NMDA receptor activation leads to excessive calcium entry, activating degradative enzymes that damage cellular structures and lead to apoptosis.

### Mechanisms

Mitochondrial Dysfunction: Excessive calcium can overwhelm mitochondrial calcium buffering capacity, leading to the production of reactive oxygen species (ROS) and cell death.
Caspase Activation: Calcium influx can activate caspases, a family of proteases involved in apoptosis.

### Exogenous Drugs

Memantine: A low-affinity NMDA receptor antagonist that prevents excitotoxicity by reducing excessive receptor activation.
Amantadine: Another NMDA receptor antagonist that offers neuroprotective effects, particularly in Parkinson's disease and drug-induced dyskinesias.

Another class of drugs regulate the calcium system. The calcium system modulates neuroplasticity by regulating calcium influx through voltage-gated channels, calcium release from intracellular stores, and calcium buffering. Drugs like calcium channel blockers, SERCA inhibitors, and calcium chelators impact calcium signaling pathways, affecting synaptic strength and neuronal health.

E) Exogenous Drugs that Modify Calcium-Based Plasticity

### These Include Calcium Channel Modulators Such as:

Nifedipine which acts as a dihydropyridine calcium channel blocker that inhibits L-type calcium channels, used to treat hypertension and angina.
Verapamil which acts as a non-dihydropyridine calcium channel blocker that inhibits L-type calcium channels, also used for hypertension and arrhythmias.
Gabapentin which acts to inhibit voltage-gated calcium channels (specifically the α2δ subunit) and is used for neuropathic pain and epilepsy.

### Calcium Release Modulators Such as:

Ryanodine which acts to bind to ryanodine receptors (RyRs) on the sarcoplasmic/endoplasmic reticulum, modulating calcium release.
Caffeine which acts to sensitize ryanodine receptors, increasing calcium release from the sarcoplasmic reticulum.
Dantrolene which acts to inhibit ryanodine receptors, reducing calcium release, and is used to treat malignant hyperthermia.

### Calcium Pump Modulators Such as:

Thapsigargin which acts to inhibit the sarco/endoplasmic reticulum sarcoplasmic/endoplasmic reticulum Ca²⁺-ATPase (SERCA), preventing calcium reuptake into the endoplasmic reticulum.
Cyclopiazonic Acid which acts to inhibit SERCA, leading to elevated cytosolic calcium levels.

### Calcium Buffering Agents Such as:

BAPTA-AM which acts as a calcium chelator that buffers intracellular calcium, preventing calcium-dependent signaling pathways.
EGTA which acts as a calcium chelator with a higher selectivity for calcium over magnesium, used to buffer extracellular calcium.

### Calcium-Sensing Receptor Modulators Such as:

Cinacalcet which acts as a calcimimetic that enhances the sensitivity of the calcium-sensing receptor (CaSR), used to treat hyperparathyroidism.
NPS 2143 which acts as a calcilytic that acts as an antagonist to the calcium-sensing receptor, potentially increasing parathyroid hormone (PTH) release.

### Methods of Modifying Calcium-Based Plasticity

Calcium Influx through Voltage-Gated Calcium Channels (VGCCs)

Voltage-gated calcium channels (VGCCs) play a critical role in calcium influx upon membrane depolarization, influencing various cellular processes including neurotransmitter release and gene expression.

### Process

Depolarization-Induced Calcium Influx: Membrane depolarization opens VGCCs, allowing calcium ions to enter the cell and initiate downstream signaling pathways.

### Channels

L-type VGCCs: Predominantly found in muscle cells and neurons, involved in prolonged calcium influx.
N-type and P/Q-type VGCCs: Found in neurons, primarily involved in neurotransmitter release.

### Exogenous Drugs

Nifedipine which acts to block L-type calcium channels, reducing calcium influx.
Verapamil which acts to block L-type calcium channels, used to control heart rate and blood pressure.
Gabapentin which acts to inhibit the α2δ subunit of VGCCs, reducing calcium influx and neurotransmitter release.
   Calcium Release from Intracellular Stores Calcium release from intracellular stores, primarily the endoplasmic reticulum (ER) or sarcoplasmic reticulum (SR), is a major source of calcium signaling within cells.

### Process

IP3-Mediated Release: Inositol trisphosphate (IP3) acts to bind to its receptor (IP3R) on the ER, triggering calcium release.
Ryanodine Receptor-Mediated Release: Ryanodine receptors (RyRs) on the ER/SR release calcium in response to various stimuli.

### Channels/Receptors

IP3 Receptors (IP3Rs) acts to mediate calcium release in response to IP3.
Ryanodine Receptors (RyRs) acts to mediate calcium release from the SR/ER in response to stimuli like caffeine or ryanodine.

### Exogenous Drugs

Ryanodine which acts to modulate RyR function, either stabilizing the open state at low concentrations or blocking it at higher concentrations.
Caffeine which acts to sensitize RyRs, increasing calcium release.
Dantrolene which acts to inhibit RyRs, reducing calcium release, used in conditions like malignant hyperthermia.
   Calcium Reuptake into Intracellular Stores

Reuptake of calcium into intracellular stores is essential for maintaining calcium homeostasis and terminating calcium signaling.

### Process

SERCA Pump Activity: The sarco/endoplasmic reticulum Ca²⁺-ATPase (SERCA) actively transports calcium from the cytosol back into the ER/SR, reducing cytosolic calcium levels.
Enzymes/Transporters
SERCA: A pump that reuptakes calcium into the ER/SR, maintaining low cytosolic calcium levels.

### Exogenous Drugs

Thapsigargin which inhibits SERCA, preventing calcium reuptake and increasing cytosolic calcium levels.
Cyclopiazonic Acid which inhibits of SERCA, leading to sustained elevations in cytosolic calcium.

### Calcium Buffering and Chelation

Calcium buffering and chelation involve the use of molecules that bind free calcium ions, preventing them from participating in signaling pathways.

### Process

Calcium Binding: Calcium buffers or chelators bind to free calcium ions, reducing their availability for signaling and helping to maintain cellular calcium homeostasis.

### Buffers/Chelators

BAPTA (1, 2-bis(o-aminophenoxy) ethane-N,N,N′,N′-tetraacetic acid) which acts as a synthetic calcium chelator with a high affinity for calcium, used to buffer intracellular calcium.
EGTA (ethylene glycol-bis(β-aminoethyl ether)-N,N,N′,N′-tetraacetic acid) which acts as a calcium chelator with higher selectivity for calcium over magnesium, often used in extracellular environments.

### Exogenous Drugs

BAPTA-AM which is a membrane-permeable form of BAPTA, used to buffer intracellular calcium.
EGTA which is used to buffer extracellular calcium and prevent calcium-dependent processes.

### Modulation of Calcium-Sensing Receptors (CaSR)

Calcium-sensing receptors (CaSR) are G-protein-coupled receptors that detect extracellular calcium levels and modulate various physiological processes, including parathyroid hormone (PTH) secretion.

### Process

Calcium Detection: CaSRs detect extracellular calcium levels and modulate downstream signaling pathways that affect processes like hormone secretion.

### Receptors

Calcium-Sensing Receptors (CaSR): G-protein-coupled receptors that sense extracellular calcium and regulate PTH secretion.

### Exogenous Drugs

Cinacalcet which is a calcimimetic that enhances the sensitivity of CaSR, reducing PTH secretion.
NPS 2143 which is a calcilytic that antagonizes CaSR, potentially increasing PTH secretion.

Another class of drugs regulate cytoskeletal restructuring. Cytoskeletal restructuring involves modulating the actin and microtubule dynamics that underlie neuronal shape, spine formation, and synaptic plasticity. Drugs such as cytochalasins and microtubule stabilizers target cytoskeletal components to promote or inhibit structural changes in neurons, influencing connectivity and neuroplasticity.

F) Exogenous Drugs that Modify Cytoskeletal Plasticity

### Actin Modulators Such as:

Cytochalasin D which acts to bind to the barbed end of actin filaments, preventing polymerization and leading to depolymerization.
Latrunculin A which acts to bind to actin monomers (G-actin), preventing their polymerization into filaments (F-actin).
Phalloidin which acts to Bind and stabilize F-actin, preventing depolymerization and promoting actin filament stability.

### Microtubule Modulators Such as:

Paclitaxel (Taxol) which acts to stabilize microtubules by binding to the β-tubulin subunit, preventing depolymerization and leading to cell cycle arrest.
Colchicine which acts to bind to tubulin, inhibiting microtubule polymerization and leading to depolymerization.
Vinblastine which acts to inhibit microtubule assembly by binding to tubulin, used in cancer therapy to disrupt mitotic spindle formation.

### Intermediate Filament Modulators Such as:

Withaferin A which acts to disrupt vimentin filaments, leading to the reorganization of intermediate filaments and inhibition of cell migration.
Simvastatin which has been shown to disrupt intermediate filament assembly, particularly of vimentin, potentially affecting cell motility and structural integrity.

### Rho GTPase Modulators Such as:

C3 Transferase which acts to inhibit Rho GTPase by ADP-ribosylation, leading to cytoskeletal disruption and loss of stress fibers.
Y-27632 which acts to inhibit ROCK (Rho-associated protein kinase), leading to decreased actin-myosin contractility and reduced stress fiber formation.

### Myosin II Modulators Such as

Blebbistatin which acts to inhibit non-muscle myosin II ATPase activity, reducing actin-myosin interactions and affecting cell contraction and motility.
ML-7 which acts to inhibit myosin light chain kinase (MLCK), reducing phosphorylation of myosin light chains and decreasing cell contractility.

### Methods of Modifying Cytoskeletal Plasticity

### Actin Dynamics

Actin filaments (F-actin) are critical for cell shape, motility, and various cellular processes, and their dynamics are regulated by polymerization and depolymerization.

### Process

Polymerization: Actin monomers (G-actin) polymerize to form filamentous actin (F-actin).
Depolymerization: F-actin disassembles back into G-actin monomers, allowing for cytoskeletal reorganization.

### Proteins/Enzymes

Actin: The primary protein involved in filament formation and turnover.
Cofilin: Binds to ADP-actin filaments, accelerating disassembly.
Profilin: Binds to G-actin and facilitates its addition to the growing end of F-actin.

### Exogenous Drugs

Cytochalasin D which binds to the barbed end of actin filaments, preventing polymerization.
Latrunculin A which binds to G-actin, preventing its polymerization into F-actin.
Phalloidin which stabilizes F-actin, preventing depolymerization.

### Microtubule Dynamics

Microtubules are essential for maintaining cell structure, intracellular transport, and cell division, with their dynamics regulated by polymerization and depolymerization.

### Process

Polymerization: Tubulin dimers (α- and β-tubulin) polymerize to form microtubules.
Depolymerization: Microtubules disassemble into tubulin dimers, allowing for cytoskeletal rearrangement.

### Proteins/Enzymes

Tubulin: The building block of microtubules.
Kinesin and Dynein: Motor proteins that move along microtubules, transporting cellular cargo.

### Exogenous Drugs

Paclitaxel (Taxol) which acts stabilize microtubules, preventing depolymerization.
Colchicine which binds to tubulin, inhibiting microtubule polymerization.
Vinblastine which acts to inhibit microtubule assembly, disrupting mitotic spindle formation.

### Intermediate Filament Dynamics

Intermediate filaments provide structural support and are involved in maintaining cell integrity, with their dynamics regulated by assembly and disassembly.

### Process

Assembly: Intermediate filament proteins polymerize to form filaments that provide mechanical support.
Disassembly: Intermediate filaments depolymerize, allowing for cytoskeletal remodeling.

### Proteins/Enzymes

Vimentin: A type of intermediate filament protein involved in maintaining cell shape and integrity.
Keratin: An intermediate filament protein found in epithelial cells, providing structural support.

### Exogenous Drugs

Withaferin A which acts to disrupt vimentin filaments, affecting cell migration and structural integrity.
Simvastatin which acts to disrupt intermediate filament assembly, particularly vimentin, influencing cell motility.

### Regulation by Rho GTPases

Rho GTPases are molecular switches that regulate cytoskeletal dynamics, influencing actin filament formation, microtubule stability, and cell motility.

### Process

Activation/Inactivation: Rho GTPases cycle between an active GTP-bound state and an inactive GDP-bound state, controlling cytoskeletal organization.

### Proteins/Enzymes

RhoA which acts to promote the formation of stress fibers and focal adhesions.
Rac1 which acts to promote the formation of lamellipodia, essential for cell migration.
Cdc42 which acts to regulate the formation of filopodia, involved in sensing the cellular environment.

### Exogenous Drugs

C3 Transferase which acts to inhibit Rho GTPase, leading to cytoskeletal disruption.
Y-27632 which acts to inhibit ROCK, a downstream effector of RhoA, reducing stress fiber formation.

### Actin-Myosin Interactions

Actin-myosin interactions are crucial for cellular contractility, motility, and cytokinesis, driven by the ATP-dependent movement of myosin along actin filaments.

### Process

Contraction: Myosin II motors interact with actin filaments, leading to cell contraction and motility.

### Proteins/Enzymes

Myosin II: A motor protein that interacts with actin to generate contractile force.
Myosin Light Chain Kinase (MLCK): Phosphorylates myosin light chains, enhancing myosin's interaction with actin.

Still yet another class of drugs effect epigenetic modulation. Epigenetic modulation involves altering chromatin structure and gene expression through histone modifications, DNA methylation, and chromatin remodeling. Compounds like HDAC inhibitors, DNMT inhibitors, and bromodomain inhibitors influence neuroplasticity by regulating transcription and gene activity.

G) Exogenous Drugs that Modify Chromatin Restructuring

### Histone Modifications Such as:

HDAC Inhibitors: e.g., Vorinostat (SAHA), Valproic Acid—which act to prevent deacetylation, maintaining gene activation.
HAT Activators: e.g., C646—which acts to enhance HAT activity, promoting gene activation.
EZH2 Inhibitors: e.g., Tazemetostat—which acts to inhibit EZH2, reducing H3K27 methylation and potentially reactivating silenced genes.
LSD1 Inhibitors: e.g., Tranylcypromine—which acts to inhibit LSD1, preventing demethylation of H3K4 and maintaining gene activation.
Aurora Kinase Inhibitors: e.g., Alisertib—which acts to target Aurora B kinase, potentially affecting histone phosphorylation.
ATM/ATR Inhibitors: e.g., Caffeine—which acts to inhibit ATM/ATR, affecting phosphorylation in DNA damage response.
Proteasome Inhibitors: e.g., Bortezomib—which acts to inhibit the proteasome, affecting the degradation of ubiquitinated proteins.
DUB Inhibitors: e.g., WP1130—which acts to target deubiquitinases (DUBs), potentially affecting histone ubiquitination.
SUMOylation Inhibitors: e.g., Ginkgolic Acid—which has been shown to inhibit SUMOylation processes in some studies.

### DNA Methylation Such as:

DNMT Inhibitors: e.g., Azacitidine (Vidaza), Decitabine—which acts to inhibit DNA methyltransferases, leading to DNA demethylation and potential reactivation of silenced genes.
Vitamin C which acts to enhance the activity of TET enzymes, promoting DNA demethylation.

### Chromatin Remodeling Such as:

Bromodomain Inhibitors: e.g., JQ1—which acts to target bromodomains of chromatin remodelers like BRD4, influencing gene expression.
Curcumin which affects chromatin remodeling complexes like SWI/SNF, though its exact mechanism remains under investigation.

### Chromatin Looping Such as:

Thalidomide which acts to target CTCF and Cohesin interactions, affecting chromatin looping and gene regulation.
BET Inhibitors: e.g., JQ1—which acts to indirectly affect looping by disrupting enhancer-promoter interactions.

### Histone Variants Such as:

Histone Deacetylase Inhibitors: e.g., Valproic Acid—which acts to promote the incorporation of H₂A.Z by altering chromatin structure.
Nicotine which has been shown to affect the deposition of certain histone variants like H3.3, impacting gene regulation.

### Polycomb/Trithorax Group Proteins Such as:

EZH2 Inhibitors: e.g., Tazemetostat—which acts to target EZH2 in PRC2, reducing H3K27 methylation.
BMI-1 Inhibitors: e.g., PTC-209—which acts to target BMI-1, a component of PRC1, affecting gene repression.
Menin-MLL Inhibitors: e.g., MI-503—which acts to disrupt the interaction between Menin and MLL, affecting H3K4 methylation and gene activation.

### Methylation and Gene Activation

### Types of Chromatin Restructuring

The main types of chromatin restructuring include:

### Histone Modifications

Histone modifications involve the addition or removal of chemical groups to histone proteins, influencing chromatin structure and gene expression.

### Acetylation

Process: Addition of acetyl groups to lysine residues on histones, generally leading to gene activation by loosening chromatin.
Enzymes:
   Histone Acetyltransferases (HATs): e.g., p300/CBP, GCN5.
   Histone Deacetylases (HDACs): e.g., HDAC1, SIRT1.
Exogenous Drugs:
   HDAC Inhibitors: e.g., Vorinostat (SAHA), Valproic Acid—which acts to prevent deacetylation, maintaining gene activation.
   HAT Activators: e.g., C646—which acts to enhance HAT activity, promoting gene activation.

### Methylation

Process: Addition of methyl groups to lysine or arginine residues on histones, influencing gene activation or repression depending on the site.
Enzymes:
   Histone Methyltransferases (HMTs): e.g., SETD1 (H3K4me3), EZH2 (H3K27me3).
   Histone Demethylases (HDMs): e.g., LSD1 (H3K4me1/2), JARID1 (H3K4me3 demethylase).
Exogenous Drugs:
   EZH2 Inhibitors: e.g., Tazemetostat—which acts to inhibit EZH2, reducing H3K27 methylation and potentially reactivating silenced genes.
   LSD1 Inhibitors: e.g., Tranylcypromine—which acts to inhibit LSD1, preventing demethylation of H3K4 and maintaining gene activation.

### Phosphorylation

Process: Addition of phosphate groups to serine or threonine residues on histones, often associated with chromatin condensation and DNA repair.
Enzymes:
   Kinases: e.g., Aurora B kinase (H3S10ph).
   Phosphatases: e.g., PP1 (H3S10ph dephosphorylation).
Exogenous Drugs:
   Aurora Kinase Inhibitors: e.g., Alisertib—which acts to target Aurora B kinase, potentially affecting histone phosphorylation.
   ATM/ATR Inhibitors: e.g., Caffeine—which acts to inhibit ATM/ATR, affecting phosphorylation in DNA damage response.

### Ubiquitination

Process: Addition of ubiquitin to lysine residues on histones, which can signal for degradation or alter chromatin structure.
Enzymes:
   E3 Ubiquitin Ligases: e.g., RNF20/40 (H2BK120ub).
   Deubiquitinases (DUBs): e.g., USP22 (removes ubiquitin from H2B).
Exogenous Drugs:
   Proteasome Inhibitors: e.g., Bortezomib—which acts to inhibit the proteasome, affecting the degradation of ubiquitinated proteins.
   DUB Inhibitors: e.g., WP1130—which acts to target DUBs, potentially affecting histone ubiquitination.

### Sumoylation

Process: Addition of SUMO proteins to histones, typically associated with gene repression.
Enzymes:
   SUMO E3 Ligases: e.g., PIAS1.
   SUMO Proteases: e.g., SENP1 (removes SUMO from histones).
Exogenous Drugs:
   SUMOylation Inhibitors: e.g., Ginkgolic Acid—which acts to inhibit SUMOylation processes in some studies.

### DNA Methylation

DNA methylation typically involves adding a methyl group to cytosine bases within CpG dinucleotides, leading to gene repression.

### Process

Methylation of cytosines in CpG islands, typically leading to transcriptional silencing.

### Enzymes

DNA Methyltransferases (DNMTs): e.g., DNMT1 (maintenance methylation), DNMT3A/B (de novo methylation).
DNA Demethylases: TET proteins (e.g., TET1) convert 5-methylcytosine to 5-hydroxymethylcytosine, facilitating DNA demethylation.

### Exogenous Drugs

DNMT Inhibitors: e.g., Azacitidine (Vidaza), Decitabine—which act to inhibit DNA methyltransferases, leading to DNA demethylation and potential reactivation of silenced genes.
Vitamin C which acts to enhance the activity of TET enzymes, promoting DNA demethylation.

### Chromatin Remodeling

Chromatin remodeling involves the repositioning or restructuring of nucleosomes, allowing access to DNA for transcription.

### Process

ATP-dependent complexes move, eject, or restructure nucleosomes to expose or hide regulatory regions of DNA.

### Enzymes

SWI/SNF Complex: Involved in nucleosome sliding and ejection, facilitating transcriptional activation.
ISWI Complex: Repositions nucleosomes to maintain chromatin structure and repress transcription.

### Exogenous Drugs

Bromodomain Inhibitors: e.g., JQ1—which acts to target bromodomains of chromatin remodelers like BRD4, influencing gene expression.
Curcumin which acts to affect chromatin remodeling complexes like SWI/SNF, though its exact mechanism remains under investigation.

### Chromatin Looping

Chromatin looping brings distant genomic regions into close proximity, often regulating gene expression by enhancing or insulating interactions.

### Process

DNA loops form, bringing enhancers into contact with promoters or insulating regions to regulate gene activity.

### Enzymes/Proteins

CTCF (CCCTC-binding factor): A key protein in forming chromatin loops, often at insulator sites.
Cohesin Complex: Works with CTCF to stabilize loops.

### Exogenous Drugs

Thalidomide which acts to target CTCF and Cohesin interactions, affecting chromatin looping and gene regulation.
BET Inhibitors: e.g., JQ1—which acts to indirectly affect looping by disrupting enhancer-promoter interactions.

### Histone Variants

Histone variants replace standard histones in nucleosomes, altering chromatin structure and gene expression.

### Process

Incorporation of histone variants into nucleosomes to modify chromatin properties and gene regulation.

### Enzymes/Chaperones

H2A.Z: Variant associated with gene activation.
MacroH2A: Variant associated with gene repression and X-chromosome inactivation.
HIRA Chaperone: Involved in the incorporation of H3.3, a variant of H3 associated with active chromatin.

### Exogenous Drugs

Histone Deacetylase Inhibitors: e.g., Valproic Acid—which acts to promote the incorporation of H2A.Z by altering chromatin structure.
Nicotine which acts to affect the deposition of certain histone variants like H3.3, impacting gene regulation.

### Polycomb/Trithorax Group Proteins

These protein complexes maintain long-term gene repression or activation through chromatin modifications.

### Polycomb Group (PcG) Proteins

Process: Maintain gene repression through histone modifications and chromatin compaction.
Enzymes/Complexes:
   PRC2: Methylates H3K27 (H3K27me3) to repress genes.
   PRC1: Ubiquitinates H2AK119 to maintain repressed chromatin.
Exogenous Drugs:
   EZH2 Inhibitors: e.g., Tazemetostat—which acts to target EZH2 in PRC2, reducing H3K27 methylation.
   BMI-1 Inhibitors: e.g., PTC-209—which acts to target BMI-1, a component of PRC1, affecting gene repression.

### Trithorax Group (TrxG) Proteins

Process: Maintain gene activation by counteracting Polycomb group proteins and modifying histones.
Enzymes/Complexes:
   MLL Complex: Methylates H3K4 (H3K4me3) which act to promote gene activation.
Exogenous Drugs:
   Menin-MLL Inhibitors: e.g., MI-503—which acts to disrupt the interaction between Menin and MLL, affecting H3K4 methylation and gene activation.

### Molecular Signaling Pathways

Molecular signaling pathways modulate neuroplasticity through protein kinases, phosphatases, and secondary messengers like cAMP and calcium. Drugs that target these pathways, such as kinase inhibitors and phosphodiesterase inhibitors, influence cell growth, metabolism, and synaptic plasticity.

Also useful are drugs that modify molecular signaling pathways. Molecular signaling pathways modulate neuroplasticity through protein kinases, phosphatases, and secondary messengers like CAMP and calcium. Drugs that target these pathways, such as kinase inhibitors and phosphodiesterase inhibitors, influence cell growth, metabolism, and synaptic plasticity.

H) Exogenous Drugs that Modify Molecular Signaling Pathways

### Protein Kinase Modulators Such as:

Imatinib which acts as a tyrosine kinase inhibitor used to treat chronic myeloid leukemia by targeting the BCR-ABL fusion protein.
Sorafenib which acts as a multi-kinase inhibitor that targets RAF kinase, Vascular endothelial growth factor receptors (VEGFR), and Platelet-derived growth factor receptors (PDGFR), used in cancer therapy.
Sirolimus (Rapamycin) which acts as an mTOR inhibitor that suppresses immune responses and is used as an immunosuppressant in organ transplantation.

### Protein Phosphatase Modulators Such as:

Okadaic Acid which acts to inhibit protein phosphatases 1 and 2A, leading to hyperphosphorylation of various substrates.
Cyclosporine which acts to inhibit calcineurin, a protein phosphatase involved in T-cell activation, used as an immunosuppressant.
Fostriecin which acts to inhibit protein phosphatase 2A (PP2A), leading to increased phosphorylation of proteins involved in cell cycle regulation.

### Secondary Messenger Modulators Such as:

Forskolin which acts to activate adenylyl cyclase, increasing cAMP levels, and is used in research to study cAMP-mediated pathways.
Caffeine which acts to inhibit phosphodiesterase (PDE), preventing cAMP breakdown, and also affects calcium release from intracellular stores.
Lithium which acts to inhibit inositol monophosphatase, affecting the phosphoinositide signaling pathway and reducing the levels of inositol trisphosphate (IP3).

Another class of useful drugs includes drugs that modify structural plasticity. Structural plasticity refers to changes in the physical architecture of neurons, such as dendritic spine remodeling, synaptogenesis, and axonal sprouting. Compounds like BDNF, lithium, and neurotrophic factors modulate these processes to enhance connectivity, learning, and repair after injury.

### Methods of Modifying Molecular Signaling Pathways

### Protein Kinases

Protein kinases are enzymes that transfer a phosphate group from ATP to a specific amino acid residue on a target protein, regulating various cellular processes such as metabolism, cell division, and signal transduction.

### Process

Phosphorylation: The transfer of a phosphate group to serine, threonine, or tyrosine residues on target proteins, altering their activity, localization, or interaction with other proteins.

### Types of Kinases

Tyrosine Kinases: Phosphorylate tyrosine residues on target proteins, often involved in growth factor signaling (e.g., EGFR, BCR-ABL).
Serine/Threonine Kinases: Phosphorylate serine or threonine residues, involved in pathways such as MAPK, AKT, and mTOR.
Dual-Specificity Kinases: Can phosphorylate both tyrosine and serine/threonine residues (e.g., MEK1/2 in the MAPK pathway).

### Exogenous Drugs

Imatinib which acts to inhibit BCR-ABL, a tyrosine kinase involved in chronic myeloid leukemia.
Sorafenib which act to inhibit multiple kinases, including RAF, VEGFR, and PDGFR, used in cancer treatment.
Sirolimus (Rapamycin) which acts to inhibit mTOR, a key regulator of cell growth and metabolism.

### Protein Phosphatases

Protein phosphatases are enzymes that remove phosphate groups from phosphorylated proteins, reversing the action of protein kinases and regulating the phosphorylation state of proteins.

### Process

Dephosphorylation: The removal of a phosphate group from phosphorylated serine, threonine, or tyrosine residues, often leading to deactivation of the protein or changes in its function.

### Types of Phosphatases

Serine/Threonine Phosphatases: Dephosphorylate serine or threonine residues (e.g., PP1, PP2A).
Tyrosine Phosphatases: Dephosphorylate tyrosine residues (e.g., PTP1B).
Dual-Specificity Phosphatases: Can dephosphorylate both tyrosine and serine/threonine residues (e.g., MKP-1).

### Exogenous Drugs

Okadaic Acid which acts to inhibit protein phosphatases 1 and 2A, leading to increased phosphorylation of various substrates.
Cyclosporine which acts to inhibit calcineurin, a protein phosphatase involved in T-cell activation.
Fostriecin which acts to inhibit PP2A, affecting the phosphorylation of proteins involved in cell cycle regulation.

### Secondary Messengers

Secondary messengers are intracellular signaling molecules released by cells in response to exposure to extracellular signaling molecules (first messengers), such as hormones or neurotransmitters. They amplify the signal and lead to various cellular responses.

### Common Secondary Messengers

CAMP (Cyclic Adenosine Monophosphate): A secondary messenger produced by adenylyl cyclase in response to G-protein-coupled receptor (GPCR) activation.
cGMP (Cyclic Guanosine Monophosphate): Produced by guanylyl cyclase, often in response to nitric oxide (NO) signaling.
IP3 (Inositol Trisphosphate): Generated by the cleavage of phosphatidylinositol 4,5-bisphosphate (PIP2) and involved in calcium release from the endoplasmic reticulum.
DAG (Diacylglycerol): Works in conjunction with IP3 to activate protein kinase C (PKC).

### Process

Signal Amplification: Secondary messengers amplify the signal received by receptors, leading to the activation of various downstream signaling pathways.

### Exogenous Drugs

Forskolin which acts to activate adenylyl cyclase, increasing cAMP levels.
Caffeine which acts to inhibit phosphodiesterase, preventing the breakdown of cAMP and cGMP.
Lithium which acts to inhibit inositol monophosphatase, affecting the phosphoinositide signaling pathway and reducing IP3 levels.

### CAMP/PKA Pathway

The cAMP/PKA pathway is a key signaling cascade that regulates various cellular functions, including metabolism, gene expression, and cell growth.

### Process

Activation of Adenylyl Cyclase: G-protein-coupled receptors (GPCRs) activate adenylyl cyclase, which converts ATP to CAMP.
PKA Activation: cAMP binds to the regulatory subunits of protein kinase A (PKA), releasing and activating the catalytic subunits.

### Enzymes/Proteins

Adenylyl Cyclase: Converts ATP to CAMP.
Protein Kinase A (PKA): A serine/threonine kinase activated by cAMP.

### Exogenous Drugs

Forskolin which acts to activate adenylyl cyclase, increasing cAMP levels.
Caffeine which acts to inhibit phosphodiesterase, preventing cAMP breakdown and prolonging PKA activation.

### PI3K/AKT/mTOR Pathway

The PI3K/AKT/mTOR pathway is involved in regulating cell growth, proliferation, and survival, playing a critical role in cancer and metabolic diseases.

### Process

Activation of PI3K: Growth factors activate phosphoinositide 3-kinase (PI3K), leading to the production of PIP3.
AKT Activation: PIP3 recruits and activates AKT, which then phosphorylates various downstream targets.
mTOR Activation: AKT activates mTOR, promoting cell growth and protein synthesis.

### Enzymes/Proteins

PI3K: Produces PIP3, a lipid second messenger.
AKT (Protein Kinase B): A serine/threonine kinase activated by PIP3.
mTOR (Mammalian Target of Rapamycin): A central regulator of cell growth and metabolism.

### Exogenous Drugs

Sirolimus (Rapamycin) which acts to inhibit mTOR, suppressing cell growth and proliferation.
Wortmannin which acts to inhibit PI3K, blocking the activation of AKT and downstream signaling.

### Calcium/Calmodulin Pathway

Calcium ions act as a secondary messenger in various signaling pathways, often working in conjunction with calmodulin, a calcium-binding messenger protein.

### Process

Calcium Influx/Release: Calcium enters the cytosol through channels or is released from intracellular stores.
Calmodulin Activation: Calcium binds to calmodulin, leading to the activation of various enzymes and proteins.

### Proteins/Enzymes

Calmodulin: A calcium-binding protein that activates various kinases and phosphatases.
Calmodulin-Dependent Protein Kinases (CaMKs): Activated by the calcium/calmodulin complex, involved in regulating transcription and other cellular processes.

### Exogenous Drugs

W-7 which acts as an inhibitor of calmodulin, preventing the activation of CaMKs and other calmodulin-dependent processes.
KN-93 which acts to inhibit CaMKII, blocking calcium/calmodulin-dependent signaling.
   I) Exogenous Drugs that Modify Structural Plasticity

### Dendritic Spine Remodeling Modulators Such as:

BDNF (Brain-Derived Neurotrophic Factor) that acts to promote dendritic spine growth and synaptic plasticity.
Tianeptine which acts to enhance dendritic spine remodeling and synaptic plasticity, often used in the treatment of depression.
Ketamine which acts to induce dendritic spine formation and enhances synaptic strength, particularly in the context of depression treatment.

### Synaptogenesis Modulators Such as:

Estrogen which act to promote synaptogenesis in the hippocampus and cortex, with implications for memory and cognition.
IGF-1 (Insulin-like Growth Factor 1) which acts to stimulate synapse formation and neuronal survival, with potential applications in neurodegenerative diseases.
Lithium which acts to enhance synaptogenesis and neurogenesis, often used as a mood stabilizer in bipolar disorder.

### Axonal Sprouting and Pruning Modulators Such as:

NGF (Nerve Growth Factor) which acts to promote axonal growth and sprouting, particularly in peripheral neurons.
Corticosteroids which acts to inhibit axonal sprouting and induce pruning, often used to reduce inflammation.
Rho Kinase Inhibitors which acts to promote axonal regeneration and sprouting by inhibiting pathways that limit growth.

### Methods of Modifying Structural Plasticity

### Dendritic Spine Remodeling

Dendritic spines are small protrusions on the dendrites of neurons that receive synaptic inputs. Their remodeling is crucial for synaptic plasticity, learning, and memory.

### Process

Spine Formation and Maturation: Dendritic spines can form, enlarge, or retract in response to synaptic activity, which is essential for the plasticity of neural circuits.
Structural Changes: Spine remodeling involves changes in the actin cytoskeleton, as well as the recruitment or removal of synaptic proteins.

### Proteins/Enzymes

BDNF (Brain-Derived Neurotrophic Factor): Promotes spine growth and stabilization.
Actin: The primary cytoskeletal component involved in spine formation and restructuring.
Synaptic Scaffolding Proteins: e.g., PSD-95, which organizes receptors and signaling molecules at the postsynaptic density.

### Exogenous Drugs

BDNF: which acts to promote dendritic spine growth and synaptic plasticity.
Tianeptine which acts to enhance dendritic spine remodeling, particularly in response to stress and depression.
Ketamine which acts to induce rapid dendritic spine formation and increases synaptic strength.

### Synaptogenesis

Synaptogenesis refers to the formation of new synapses between neurons, which is essential for the development of neural circuits and the maintenance of cognitive functions.

### Process.

Synapse Formation: Involves the contact between axons and dendrites, followed by the recruitment of synaptic vesicles, receptors, and scaffolding proteins.
Maturation and Stabilization: Newly formed synapses undergo maturation and stabilization, often requiring activity-dependent signaling.

### Proteins/Enzymes

Synapsins: Regulate the availability of synaptic vesicles for release.
Neuroligins/Neurexins: Cell adhesion molecules involved in synapse formation and differentiation.
BDNF: Also involved in promoting synapse formation and stability.

### Exogenous Drugs

Estrogen which acts to enhance synaptogenesis, particularly in the hippocampus and cortex.
IGF-1 which acts to promote synapse formation and neuronal survival, with neuroprotective effects.
Lithium which acts to facilitate synaptogenesis and neurogenesis, contributing to its mood-stabilizing effects.

### Axonal Sprouting and Pruning

Axonal sprouting involves the growth of new axonal branches, while pruning refers to the selective elimination of axonal connections. These processes are crucial for the refinement of neural circuits during development and after injury.

### Process

Axonal Sprouting: New axonal branches grow from existing neurons, often in response to injury or changes in neural activity.
Axonal Pruning: Unnecessary or weak axonal branches are selectively eliminated to refine neural circuits.

### Proteins/Enzymes

NGF (Nerve Growth Factor): Promotes axonal growth and sprouting, particularly in the peripheral nervous system.
Semaphorins: Involved in axonal guidance, repulsion, and pruning.
Rho GTPases: Regulate cytoskeletal dynamics involved in axonal growth and pruning.

### Exogenous Drugs

NGF which acts to promote axonal growth and sprouting, particularly following injury.
Corticosteroids which act to inhibit axonal sprouting and promote pruning, often used to reduce inflammation.
Rho Kinase Inhibitors which act to enhance axonal sprouting and regeneration by inhibiting growth-constraining pathways.

### Synaptic Pruning

Synaptic pruning is the process of eliminating weaker synaptic connections, which is essential for the development and refinement of neural circuits.

### Process

Activity-Dependent Pruning: Less active synapses are tagged for removal, often involving microglial cells that engulf the synapses.
Molecular Signaling: Involves signals like complement proteins and major histocompatibility complex (MHC) molecules that mark synapses for pruning.

### Proteins/Enzymes

Complement Proteins: Mark synapses for elimination by microglia.
MHC Molecules: Involved in the recognition and tagging of synapses for pruning.
Microglia: Immune cells in the brain that play a key role in synaptic pruning.

### Exogenous Drugs

Minocycline an antibiotic which acts to inhibit microglial activation, potentially reducing excessive synaptic pruning.
Ketamine which acts to promote dendritic spine formation, and which also may influence synaptic pruning processes.

Still another class of drugs useful in the present disclosure are drugs that influence neurotrophic factors. Neurotrophic factors like BDNF, NGF, and GDNF play critical roles in supporting neuronal survival, growth, and synaptic plasticity. Drugs that increase neurotrophic factor levels, such as SSRIs and dopamine agonists, enhance neuroplasticity and promote recovery in neurodegenerative diseases.

J) Exogenous Drugs that Influence Neurotrophic Factors

### BDNF Modulators Such as:

SSRIs (e.g., Fluoxetine) which acts to increase BDNF levels, contributing to their antidepressant effects.
Ketamine which acts to increase BDNF levels, particularly in the context of depression treatment.
7,8-Dihydroxyflavone which acts as a TrkB receptor agonist that mimics BDNF effects and promotes neuronal survival and plasticity.

### NGF Modulators Such as:

Melatonin which acts to enhance NGF production and supports neuroprotection.
Captopril which acts as an ACE inhibitor that increases NGF levels, potentially offering neuroprotective effects.
Curcumin which acts to increase NGF expression, contributing to its neuroprotective and anti-inflammatory properties.

### GDNF Modulators Such as:

Levodopa which acts to increase GDNF expression, particularly in the treatment of Parkinson's disease.
Dopamine Agonists (e.g., Ropinirole) which acts to increase GDNF levels, providing neuroprotection in Parkinson's disease.
Lithium which acts to enhance GDNF expression, supporting neurogenesis and neuroprotection.

### Brain-Derived Neurotrophic Factor (BDNF)

### BDNF Overview

BDNF is a key neurotrophic factor involved in the survival, growth, and maintenance of neurons. It plays a crucial role in synaptic plasticity, learning, and memory.

### Process

BDNF Synthesis and Secretion: BDNF is synthesized in neurons and can be released in an activity-dependent manner, promoting synaptic growth and strengthening.
TrkB Receptor Activation: BDNF binds to the TrkB receptor, triggering signaling cascades that promote neuronal survival, synaptic plasticity, and dendritic growth.

### Signaling Pathways

PI3K/AKT Pathway: Activated by TrkB, promoting cell survival and growth.
MAPK/ERK Pathway: Involved in synaptic plasticity and memory formation.
PLCγ Pathway: Leads to the release of intracellular calcium, influencing various cellular processes.

### Exogenous Drugs

SSRIs (e.g., Fluoxetine) which ats to increase BDNF levels, which contributes to their antidepressant effects.
Ketamine which acts to increase BDNF levels, facilitating fast-acting antidepressant effects.
7,8-Dihydroxyflavone which acts as a TrkB receptor agonist that mimics BDNF effects, promoting neuroprotection and plasticity.

### Nerve Growth Factor (NGF)

### NGF Overview

NGF is essential for the growth, maintenance, and survival of certain neurons, particularly in the peripheral nervous system. It also plays a role in neuroprotection and repair in the central nervous system.

### Process

NGF Synthesis and Secretion: NGF is produced by target tissues and binds to TrkA receptors on neurons, promoting survival and differentiation.
TrkA Receptor Activation: NGF binds to TrkA, initiating signaling pathways that support neuronal survival, axonal growth, and synaptic plasticity.

### Signaling Pathways

PI3K/AKT Pathway: Promotes neuronal survival and growth.
MAPK/ERK Pathway: Supports differentiation and growth of neurons.
PLCγ Pathway: Involved in calcium signaling and various cellular responses.

### Exogenous Drugs

Melatonin which acts to enhance NGF production, providing neuroprotective effects.
Captopril which acts to increase NGF levels, potentially offering neuroprotection and cognitive benefits.
Curcumin which acts to boost NGF expression, contributing to its neuroprotective properties.

### Glial-Derived Neurotrophic Factor (GDNF)

### GDNF Overview

GDNF is crucial for the survival of dopaminergic neurons and has significant implications in neurodegenerative diseases like Parkinson's Disease. It supports neuronal survival, differentiation, and regeneration.

### Process

GDNF Synthesis and Secretion: GDNF is produced by glial cells and binds to the GFRα1 receptor, forming a complex with RET tyrosine kinase, which triggers survival and growth pathways.
GFRα1/RET Receptor Activation: GDNF binds to GFRα1, which associates with RET, activating downstream signaling pathways that promote neuronal survival and repair.

### Signaling Pathways

PI3K/AKT Pathway: Promotes neuronal survival and growth.
MAPK/ERK Pathway: Involved in neurogenesis and the maintenance of neuronal function.
PLCγ Pathway: Involved in calcium signaling and cellular responses.

### Exogenous Drugs

Levodopa which acts to increase GDNF expression, particularly in Parkinson's disease, supporting dopaminergic neuron survival.
Dopamine Agonists (e.g., Ropinirole) which acts to increase GDNF levels, offering neuroprotection in Parkinson's Disease.
Lithium which acts to enhance GDNF expression, supporting neurogenesis and providing neuroprotective effects.

Also useful are drugs that influence glial cell activity. Glial cells, including astrocytes, microglia, and oligodendrocytes, support CNS functions by modulating neurotransmitter levels, immune responses, and myelination. Drugs like fluoxetine, minocycline, and clemastine target glial cells to improve neuroplasticity and repair in conditions like depression and multiple sclerosis.

K) Exogenous Drugs that Influence Glial Cell Activity

### Astrocyte Modulators Such as:

Fluoxetine which acts to increase astrocytic support functions and enhances neurogenesis.
Propentofylline which acts to modulate astrocyte activity, reducing gliosis and promoting neuroprotection.
Ibudilast which acts to suppress pro-inflammatory cytokine production by astrocytes, used in neuroinflammatory conditions.

### Microglia Modulators Such as:

Minocycline which acts to inhibit microglial activation, reducing neuroinflammation and associated neurotoxicity.
Naltrexone (Low-Dose) which acts to modulate microglial activity, reducing neuroinflammation in conditions like fibromyalgia and multiple sclerosis.
Resveratrol which acts as an anti-inflammatory compound that inhibits microglial activation and reduces oxidative stress.

### Oligodendrocyte Modulators Such as:

Clemastine which acts to enhance oligodendrocyte differentiation and promotes remyelination, investigated in multiple sclerosis.
Biotin which acts to support oligodendrocyte function and may enhance remyelination in neurodegenerative diseases.
Sodium Phenylbutyrate which acts to promote oligodendrocyte survival and differentiation, used in research for neurodegenerative diseases.

### Astrocytes

### Astrocyte Overview

Astrocytes are the most abundant glial cells in the central nervous system (CNS). They play crucial roles in maintaining the blood-brain barrier, supporting neuronal metabolism, modulating synaptic activity, and responding to injury and disease.

### Functions

Neurotransmitter Uptake and Recycling: Astrocytes take up neurotransmitters like glutamate from the synaptic cleft, preventing excitotoxicity and recycling them back to neurons.
Blood-Brain Barrier Maintenance: Astrocytes contribute to the integrity of the blood-brain barrier, regulating the passage of substances from the blood into the CNS.
Metabolic Support: Provide neurons with nutrients such as lactate, which is derived from glucose metabolism in astrocytes.
Gliosis: In response to CNS injury, astrocytes proliferate and form a glial scar, which can both protect and inhibit neural repair.

### Exogenous Drugs

Fluoxetine which acts to enhance astrocytic support functions, including neurogenesis and synaptic plasticity.
Propentofylline which acts to reduce astrocyte-mediated gliosis and promotes neuroprotection, particularly in neurodegenerative diseases.
Ibudilast which acts to inhibit pro-inflammatory cytokine production by astrocytes, reducing neuroinflammation.

### Microglia

### Microglia Overview

Microglia are the resident immune cells of the CNS, responsible for detecting and responding to pathogens, clearing debris, and modulating inflammation. They play a critical role in maintaining CNS homeostasis and responding to injury and disease.

### Functions

Immune Surveillance: Microglia continuously monitor the CNS environment, detecting pathogens and damaged cells.
Phagocytosis: Microglia engulf and digest cellular debris, apoptotic cells, and pathogens.
Inflammatory Response: Microglia produce cytokines and chemokines in response to injury or infection, modulating the inflammatory response.
Synaptic Pruning: During development, microglia are involved in synaptic pruning, a process critical for refining neural circuits.

### Exogenous Drugs

Minocycline which acts to inhibit microglial activation, reducing neuroinflammation and associated neurotoxicity in neurodegenerative diseases.
Naltrexone (Low-Dose) which acts to modulate microglial activity, reducing neuroinflammation in conditions like fibromyalgia and multiple sclerosis.
Resveratrol which has anti-inflammatory properties, inhibiting microglial activation and reducing oxidative stress.

### Oligodendrocytes

### Oligodendrocyte Overview

Oligodendrocytes are the myelinating cells of the CNS, responsible for the production and maintenance of the myelin sheath that insulates axons and facilitates rapid signal transmission.

### Functions

Myelination: Oligodendrocytes wrap their membranes around axons to form the myelin sheath, which is essential for efficient neural signaling.
Support and Protection: Myelin not only insulates axons but also provides trophic support to neurons, promoting their survival.
Remyelination: In response to demyelinating injuries, oligodendrocyte precursor cells (OPCs) differentiate into mature oligodendrocytes to restore the myelin sheath.

### Exogenous Drugs

Clemastine which acts to promote oligodendrocyte differentiation and remyelination, particularly in conditions like multiple sclerosis.
Biotin which acts to support oligodendrocyte function and myelin maintenance, with potential benefits in neurodegenerative diseases.
Sodium Phenylbutyrate which acts to enhance oligodendrocyte survival and differentiation, used in research for neurodegenerative conditions.

The aforesaid drugs and combinations thereof for modulating neuroplasticity in accordance with the present disclosure may be administered or delivered to a patient prior to, contemporaneously with or following commencement of delivery of energy to the brain of a patient receiving treatment by a variety of delivery methods including:
Oral Administration including regular and oral pills, capsules and liquids, and oral disintegrating tablets: Ingested through the gastrointestinal tract via pills, capsules, or liquids
Nasal Administration including intranasal: Delivered via the nasal cavity, often bypassing the blood-brain barrier
Intravenous (IV) or Intramuscular (IM) Injection: Delivered directly into the bloodstream or muscle for fast action
Transdermal (Patch, Gel or rub-on cream): Delivered through the skin, offering slow, sustained release
Sublingual/Buccal Administration: Absorbed via mucous membranes under the tongue or against the cheek
Inhalation: Absorbed into the bloodstream through the lungs
Intrathecal (Spinal) Delivery: Injected directly into the cerebrospinal fluid in the spine
Subcutaneous Implant or Injection: Slow-release drugs delivered under the skin
Nanoparticle and Liposomal Delivery: Encapsulated drugs designed to cross the blood-brain barrier
Intracerebral or Intracranial Delivery: Drugs injected directly into a patient's brain tissue

In addition to administration of various drugs as above outlined, certain environmental and/or behavioral changes may be employed to further enhance neuroplasticity of the brain of a patient being treated by external brain stimulation in accordance with the present disclosure including:

### Physical Exercise

Resistance Training (Weightlifting): Combining external brain stimulation sessions with resistance exercises to assess cortical changes and motor learning.

Task-Relevant Physical Activity: Pairing external brain stimulation sessions with motor coordination or sports-specific tasks to improve motor learning.

### Meditation and Focused Attention

Focused Attention Tasks: Applying external brain stimulation sessions during tasks requiring sustained attention or concentration.

### Cognitive and Emotional Conditioning

Classical Conditioning: Pairing external brain stimulation sessions with stimuli to condition the brain for positive cognitive/emotional states.

Mental Rehearsal: Using mental rehearsal or visualization alongside external brain stimulation sessions to prime the brain for neuroplasticity.

### Environmental Modulation

Novelty and Complexity: Using external brain stimulation sessions in conjunction with novel, enriched environments to enhance adaptability.

Sensory Stimulation: Pairing external brain stimulation sessions with tactile or multisensory stimulation (e.g., virtual reality).

Light Therapy: Using specific wavelengths of light (e.g., blue light) in combination with external brain stimulation sessions.

Temperature Modulation: Combining external brain stimulation with controlled heat or cold exposure (e.g., cryotherapy or infrared saunas).

### Sleep and Circadian Optimization

Rest Intervals: Investigating the effects of varying rest intervals between external brain stimulation sessions.

Napping or Controlled Sleep: Timing sleep sessions around external brain stimulation sessions to enhance neuroplasticity.

Circadian Rhythm Alignment: Aligning external brain stimulation sessions with the patient's circadian rhythm to optimize brain plasticity.

### Diet and Nutrition

Omega-3 Fatty Acids: Investigating the impact of omega-3 supplementation combined with external brain stimulation sessions.

Antioxidants: administering antioxidants such as vitamin E in conjunction with external brain stimulation sessions for neuroplasticity enhancement.

### Social Interaction and Emotional Modulation

Positive Emotional States: Inducing positive emotions (e.g., music, social interaction) to improve external brain stimulation outcomes.

Social Engagement: Pairing external brain stimulation sessions with social tasks or group activities.

Therapeutic Alliance: Enhancing external brain stimulation sessions efficacy through trust and engagement in the patient-therapist relationship.

Group Activities: Using external brain stimulation sessions in collaborative settings like problem-solving or learning tasks.

Supportive Environment: Assessing the effect of external brain stimulation sessions in nurturing social environments to enhance emotional regulation.

### Task-Specific Stimulation

Dual-Task Training: Combining external brain stimulation sessions with dual-task paradigms to assess multitasking improvement.

### Brain Stimulation Delivery Methods for Modulating Neuroplasticity

Neuroplasticity may vary based on a person's chronotype, which can be assessed through methods like self-report questionnaires (e.g., MEQ, MCTQ), sleep tracking (actigraphy), biomarkers (melatonin and cortisol), genetic testing, and sleep laboratory assessments (polysomnography). Cognitive performance tests can further determine peak times for alertness based on chronotype. Treatment timing can also influence neuroplasticity, with strategies like irregular intervals between sessions or intentional jitter (adding variability to the timing of sessions) potentially enhancing neuroplasticity. These timing variations can be applied within or between external brain stimulation sessions to optimize plasticity effects.

External brain stimulation could be delivered once per day or multiple times per day.

### Delivery Timing

### Time of Day of Treatment

Neuroplasticity may vary by ‘chronotype’ (e.g., a patient may prefer to be active in the morning or the evening). To rigorously determine a patient's chronotype, we may use self-report questionnaires like the MEQ or MCTQ, actigraphy for objective sleep tracking, and biomarkers such as melatonin or cortisol levels. Additional methods for determining a patient's chronotype include genetic testing for circadian gene variants, sleep laboratory assessments like polysomnography, and cognitive performance testing to measure peak alertness times.

### 1. Self-Report Questionnaires

Morningness-Eveningness Questionnaire (MEQ):
   The MEQ is one of the most widely used and validated tools for assessing a person's chronotype. It consists of a series of questions about sleep habits, peak alertness times, and preferred times for physical and mental activity. The score classifies individuals into morning, evening, or intermediate types.
      Reference: Horne, J. A., & Ostberg, O. (1976). A self-assessment questionnaire to determine morningness-eveningness in human circadian rhythms. International Journal of Chronobiology.
Munich ChronoType Questionnaire (MCTQ):
   The MCTQ asks more detailed questions about actual sleep behavior on workdays versus free days, considering the social constraints on sleep and wake times. It's more granular than MEO and also looks at “social jetlag,” where there's a misalignment between biological and social sleep schedules.
      Reference: Roenneberg, T., Wirz-Justice, A., & Merrow, M. (2003). Life between clocks: Daily temporal patterns of human chronotypes. Journal of Biological Rhythms.

### 2. Actigraphy (Sleep Tracking)

Objective Sleep Monitoring:
   Using an actigraph or a wearable sleep tracker (such as a Fitbit™, Oura Ring™, or WHOOP™), we can record sleep-wake patterns over several days or weeks. These devices monitor physical activity and rest to objectively determine sleep onset, wake times, and overall sleep patterns. The data provides insight into whether someone has a late or early sleep-wake cycle, helping to classify their chronotype.
      Methodology: Measure sleep onset latency, total sleep duration, and mid-sleep point. Evening chronotypes tend to have later bedtimes and wake times, with a delayed mid-sleep point compared to morning types.

### 3. Biomarkers and Hormonal Rhythms

Melatonin Secretion (Dim Light Melatonin Onset, DLMO):
   The timing of melatonin secretion is one of the most reliable markers of circadian phase. The DLMO test measures when melatonin levels rise in the evening, typically 1-2 hours before bedtime. A later DLMI suggests an evening chronotype, while an earlier DLMO suggests a morning chronotype.
      Test Procedure: Collect saliva or blood samples under dim light conditions (usually in a sleep lab or controlled setting) to detect melatonin onset.
Cortisol Awakening Response (CAR):
   Cortisol levels typically peak shortly after waking. Measuring the timing and amplitude of the cortisol awakening response can provide insight into someone's circadian rhythm. Morning types often show higher cortisol spikes earlier in the day, while evening types may have a delayed cortisol pattern.

### 4. Genetic Testing

PER3 Gene Polymorphism:
   Genetic variations, particularly in the PER3 gene, are associated with an individual's circadian preferences. Genetic tests can analyze polymorphisms in this gene to identify a predisposition for morningness or eveningness. For example, the PER3 (5/5) variant is linked with morning types, while the PER3 (4/4) variant is more common in evening types.
      Direct-to-consumer tests like 23andMe or AncestryDNA may include reports on chronotype tendencies based on genetic markers.

### 5. Sleep Laboratory Assessments

Polysomnography (PSG):
   For the most rigorous assessment, a sleep study conducted in a laboratory (polysomnography) can be used to measure various physiological indicators (brain activity, heart rate, eye movement, etc.) during sleep. This can help assess the timing and quality of sleep and detect any underlying sleep disorders that may influence circadian rhythms.

### 6. Cognitive Performance and Reaction Time Testing

Peak Cognitive Performance Tests:
   A person's peak cognitive performance can vary based on their chronotype. Performance tests administered at different times of day (e.g., reaction time, working memory tasks) can help identify when someone is most alert and productive. Morning types tend to perform better earlier in the day, while evening types show better performance later.
      Timing of Stimuli within a Treatment

Neuroplasticity may be enhanced with variance at various levels of the treatment.

### Irregular Inter Session Intervals

The timing between sessions, trains, bursts or other treatment timing elements may be intentionally irregular. For example the treatment may require 15 minutes between sessions 1, 2, and 3, but then 45 minutes between sessions 3 and 4. This spacing is non-standard to modify neuroplasticity.

### Intentional Jitter

The timing between sessions, trains, bursts or other treatment timing elements may be intentionally noisy. Irrespective of whether the intended schedule is regular or irregular, there may be additional variance introduced. As an example, the scheduled timing between sessions may be varied by up to +/−25% of the prescribed inter-session-interval. So if the interval between session 1 and 2 was determined by the protocol to be 15 minutes, the actual delivery timing may vary +/− by 3.75 minutes, thus the actual inter-session-interval being 11.25 minutes to 18.75 minutes.

The system advantageously may be used for treating depression as well as various other health conditions including, without limitations:
Eating Disorders: These disorders are characterized by abnormal or disturbed eating habits. Examples include anorexia nervosa, bulimia nervosa, and binge-eating disorder.
Trauma and Stressor-Related Disorders: These are related to the exposure to a traumatic or stressful event. Examples include post-traumatic stress disorder (PTSD) and acute stress disorder.
Obsessive-Compulsive and Related Disorders: These disorders involve obsessions (persistent, unwanted thoughts) and compulsions (repetitive behaviors). Examples are obsessive-compulsive disorder (OCD) and hoarding disorder.
Neurodevelopmental Disorders: These typically manifest early in development and feature developmental deficits that produce impairments of personal, social, academic, or occupational functioning. Examples include autism spectrum disorder (ASD), attention-deficit/hyperactivity disorder (ADHD), and learning disorders.
Dissociative Disorders: These disorders involve a disconnection between thoughts, identity, consciousness, and memory. Examples are dissociative identity disorder and dissociative amnesia.

Representative examples of treatment of various conditions in accordance with the present disclosure are set forth in the following table:

| Energy-Based | | |
|---|---|---|
| Neuromodulation | Neuroplasticity-potentiating | Condition Being |
| Treatment | Pharmaceutical Agent | Treated |
| TMS | D-cycloserine + Vyvanse | Depression, OCD, |
| | (NMDA agonist + | or suicidal |
| | dopaminergic) | ideation |
| TMS | D-Serine (NMDA agonist) | Depression |
| TMS | D-cycloserine (NMDA | Depression |
| | agonist) | |
| VNS | L-DOPA (dopaminergic) | Parkinson's disease |
| tDCS | Bupropion (dopamine | ADHA |
| | reuptake inhibitor) | |
| ECT | Gabapentin (GABAergic) | Schizophrenia |
| MST | Sertraline (serotonergic) | PTSD |
| FUS | CX-516 (AMPA modulator) | Autism spectrum |
| | | disorder |
| Optogenetics | Ifenprodil (NMDA | Stroke rehabilitation |
| | polyamine-site modulator) | |
| SCS | Pramipexole (dopamine | OCD |
| | agonist) | |
| tACS | Donepezil (cholinergic) | Mild cognitive |
| | | impairment (healthy) |
| PBM | Ketamine (NMDA | Depression |
| | antagonist) | |
| Low-field EM | D-Serine + Rapastinel | Suicidal ideation |
| | (NMDA modulators) | |
| DBS | Non-NMDA glutamate agent | Chronic pain |
| | (AMPA modulator) | |
| DBS | Rapastinel (NMDA partial | Alzheimer's disease |
| | agonist) | |

### WORKING EXAMPLES

Further features of the present disclosure will be seen from the following working examples:

### Example 1

### “ONE-D”

We treated 34 patients with depression using a combination of 20 mg lisdexamfetamine (a member of the stimulant class of dopaminergic medications) with 125 mg d-cycloserine (NMDA partial agonist) compounded into an oral dissolving tablet given as a single dose in the AM. Beginning 60 minutes after this dose, we then administered 20 sessions of TMS (using a pattern called intermittent theta-burst stimulation, iTBS, 600 pulses, which takes 3 minutes). Sessions were given every 30 minutes for a total of 9 hours and 33 minutes of treatment time, which falls within 5 half-lives of both lisdexamfetamine and d-cycloserine. TMS was administered on the Ampa One system (coil and cap, connected to a MagVenture R30 device), targeting the left dorsolateral prefrontal cortex (the standard region for treating depression). Patients returned home after treatment that day, and did not receive any additional sessions thereafter.

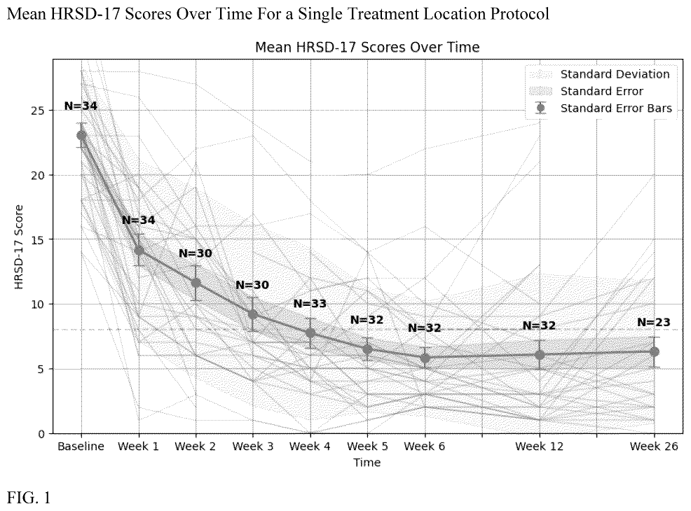

To evaluate treatment outcomes we used 3 standardized instruments commonly used in clinical trials for measuring the severity of depression symptoms: 1) A clinician-rated scale, the 17-item Hamilton Rating Scale for Depression (HRSD); 2) A patient-rated scale, the 9-item Patient Health Questionnaire (PHQ-9); 3) A second patient-rated scale, the 21-item Beck Depression Inventory-II. In addition to these three depression scales, patients filled out the Generalized Anxiety Disorder Scale (GAD-7) scale to assess changes in anxiety symptoms over time. All four of these scales were administered at a “baseline” timepoints immediately after giving the medication combination, but before starting treatment. These scales were then administered again in follow-up at a series of timepoints 1, 2, 3, 4, 5, 6, 12, and 26 weeks after the day of treatment, and the results were plotted in FIG. 1.

The standard shape of the curve for improvement in depression symptoms with conventional once-daily TMS is an exponential decay curve, spanning several weeks (since the conventional course is 36 sessions, 5 days a week, over about 7 weeks).

### Observations:

Interestingly, even though we administered all the treatments in a single day, patients showed the same exponential decay curve of improvement over the first 1-3 weeks, reaching a plateau around weeks 4-6. So this treatment regimen allowed all the sessions to be done in a single day, but only slightly accelerated the actual course of improvement. FIG. 1 plots mean HRSD-17 scores over time the 26 patients to complete treatment.

Another major benefit was in the number of patients who responded to treatment. Typically, for 20 sessions of TMS treatment, only about 20-30% of patients reach remission (defined at a certain threshold score for each scale: <8 for Hamilton, <12 for Beck, and <5 for PHQ-9) and 40-50% of patients reach response (which carries a standard definition of ≥50% improvement versus baseline). However, in our sample, 84.8% reached remission and 93.9% reached response on the Hamilton. On Beck, 79.4% reached remission and 91.2% reached response. On PHQ-9, 70.6% reached remission and 93.9% reached response.

These observed changes on HDRS-17, PHQ-9, BDI-II, and GAD-7 illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, self-awareness, impaired self-regulation, impaired insight and self-control, and suicidal ideation.

### Example 2

### Patients and Treatment:

Two patients diagnosed with Obsessive-Compulsive Disorder (OCD) were treated using a combination of 20 mg lisdexamfetamine (a stimulant-class dopaminergic medication) and 125 mg D-cycloserine (an NMDA receptor partial agonist). A single dose of each medication was taken in the morning.

### TMS Protocol:

60 minutes after the medication dose, patients received 20 sessions of TMS using a protocol called intermittent theta-burst stimulation (ITBS) (600 pulses per session, lasting 3 minutes each).
The TMS sessions were spaced 30 minutes apart over the course of 9 hours and 33 minutes, ensuring the total treatment time occurred within 5 half-lives of both lisdexamfetamine and D-cycloserine.
TMS was delivered using the Ampa One system (coil and cap), connected to a MagVenture R30 device, and targeted the left dorsolateral prefrontal cortex, the standard region for treating depression.

### Outcome Measurement:

Two standardized instruments were used to evaluate treatment outcomes:
   1. Yale-Brown Obsessive Compulsive Scale (Y-BOCS) to measure OCD severity.
   2. 9-item Patient Health Questionnaire (PHQ-9) to assess depressive symptoms.

### Results:

Patient 1:
   Y-BOCS score dropped from 32 to 7 one week after treatment, indicating remission from OCD, an exceptional outcome given that typical remission rates for TMS in OCD are in the single digits.
   PHQ-9 score decreased from 17 to 1, also indicating remission from the patient's depressive symptoms.
Patient 2:
   Y-BOCS score started at 25 and has decreased to 19, showing a positive response trajectory.
   These observed changes on Y-BOCS illustrate meaningful therapeutic effects in obsessions, compulsions, craving behaviors, urge regulation deficits, repetitive behaviors, stereotyped and habitual behaviors, and anhedonia.

### Example 3

### “TRIPLE-D”

We treated 30 patients with depression using a combination of 20 mg lisdexamfetamine (a member of the stimulant class of dopaminergic medications) with 6300 mg d-serine (NMDA agonist) given as a single dose in the AM. Beginning 60 minutes after this dose, we then administered 20 sessions of TMS. A treatment session was completed every 30 minutes for a total of 9 hours and 38 minutes of treatment time. Patients received treatment in 3 locations every 30 minutes: dorsomedial prefrontal cortex, orbitofrontal cortex, and left dorsolateral prefrontal cortex. Each treatment session lasted approximately 8 minutes and consisted of three stimulation sites. Intermittent theta-burst stimulation (iTBS; 600 pulses, ˜3 minutes) was delivered at the first and third locations, while continuous theta-burst stimulation (cTBS; 600 pulses, ˜30 seconds) was applied at the second location. TMS was administered on the Ampa One system (coil and cap, connected to a MagVenture R30 device). Two distinct Ampa coils were used within each treatment session to optimize targeting of specific brain regions. The first stimulation site was targeted using the “deep” M coil to reach subcortical structures, while the second and third sites were stimulated using the L coil, which is better suited for more superficial cortical regions. Patients returned home after treatment that day, and did not receive any additional sessions thereafter.

To evaluate treatment outcomes we used 3 standardized instruments commonly used in clinical trials for measuring the severity of depression symptoms: 1) A clinician-rated scale, the 17-item Hamilton Rating Scale for Depression (HRSD); 2) A patient-rated scale, the 9-item Patient Health Questionnaire; 3) A second patient-rated scale, the 21-item Beck Depression Inventory-II. An additional patient-rated scale was used to measure severity of anxiety symptoms: Generalized Anxiety Disorder Scale (GAD-7). All four of these scales were administered at a “baseline” timepoints immediately after giving the medication combination, but before starting treatment. These scales were then administered again in follow-up at a series of timepoints 1, 2, 3, 4, 5, 6, and 12 weeks after the day of treatment.

The standard shape of the curve for improvement in depression symptoms with conventional once-daily TMS is an exponential decay curve, spanning several weeks (since the conventional course is 36 sessions, 5 days a week, over about 7 weeks).

### Observations:

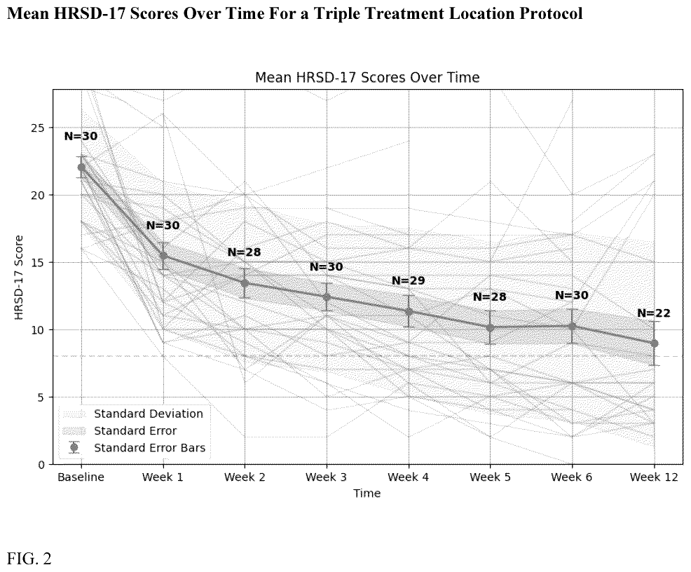

As shown in FIG. 2, mean HRSD-17 scores followed a consistent downward trajectory over time, with the steepest declines occurring during the first 1-3 weeks, followed by a more gradual tapering through weeks 4-6. Notably, this trajectory closely resembles the typical response curve seen in conventional TMS protocols, despite the accelerated nature of this intervention. By week 5, mean scores approached the remission threshold (HRSD <8, indicated by the red dashed line), and continued to decline modestly through week 12.

The observed rate of clinical improvement among participants was higher than standard TMS protocols involving 20 sessions (remission in 20-30% and response in 40-50% of patients). Our sample demonstrated markedly higher rates: 56.7% of participants achieved remission and 63.3% achieved response on the HRSD; 73.3% achieved both remission and response on the BDI-II; and 66.7% reached remission with 76.7% achieving response on the PHQ-9.

These observed changes on HDRS-17, PHQ-9, BDI-II, and GAD-7 illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, self-awareness deficits, impaired self-regulation, impaired insight and self-control, suicidal ideation.

Of note, one patient in this series tolerated treatment at amplitudes significantly below what is commonly accepted as “therapeutic” for TMS. This patient tolerated treatment over the dorsomedial prefrontal cortex at only 38% of motor threshold (MT), orbitofrontal at only 50% of MT. Despite the low treatment amplitudes, this patient still reached full remission of symptoms across the HRSD-17, PHQ-9, BDI-II, and GAD-7. This shows that patients may still have a robust treatment response when treated at less than 80% of their individualized therapeutic threshold.

### Example 4

### “ONE-A”

### Patients and Treatment:

Twenty-one patients diagnosed with generalized anxiety disorder (GAD) were treated with a combination therapy involving TMS+125 mg D-cycloserine (an NMDA receptor partial agonist). The medications were compounded into an orally dissolving tablet and administered as a single dose in the morning.

### TMS Protocol:

Sixty minutes post-medication, patients underwent an intensive course of 20 transcranial magnetic stimulation (TMS) sessions in a single day, using intermittent theta-burst stimulation (ITBS) (600 pulses per session, lasting 3 minutes each). Sessions were conducted every 30 minutes over a total treatment duration of 9 hours and 33 minutes, ensuring the pharmacological effects of both compounds remained active throughout the protocol.

TMS was administered using the Ampa One system (coil and cap), connected to a MagVenture R30 device, and targeted the dorsolateral prefrontal cortex (DLPFC)—a region implicated in anxiety regulation. Following the treatment day, no additional TMS sessions were provided.

### Outcome Measurement:

To assess treatment effectiveness, two primary standardized instruments were used to measure anxiety severity:
1. Hamilton Anxiety Rating Scale (HAM-A)—a clinician-rated assessment of anxiety symptoms.
2. Generalized Anxiety Disorder Scale (GAD-7)—a patient-reported measure of GAD severity.

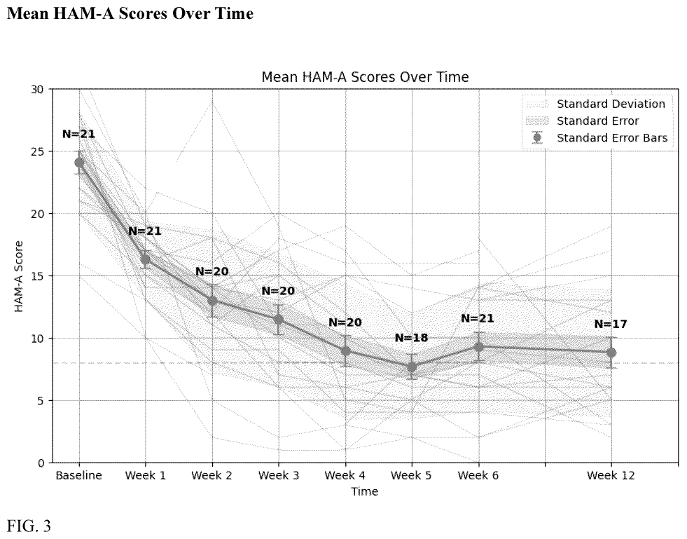

Given the frequent co-occurrence of anxiety and depression, two additional instruments were included to evaluate depressive symptoms:
1. Patient Health Questionnaire (PHQ-9)—a patient-rated measure of depressive severity.
2. Beck Depression Inventory-II (BDI-II)—a second patient-rated depression scale. Assessments were conducted at baseline (immediately following medication administration but before TMS initiation) and at follow-up intervals of 1, 2, 3, 4, 5, 6, and 12 weeks post-treatment, and the results were plotted in FIG. 3.

### Observations:

Despite the condensed treatment schedule, symptom improvement followed a progressive trajectory similar to conventional multi-week TMS protocols, with most improvement occurring within the first 3 weeks and stabilizing around weeks 4-12. (FIG. 3 illustrates mean HAM-A scores over time for the first twenty one patients who completed treatment.)

In terms of response and remission rates:
HAM-A: 71.4% remission, 81.0% response
GAD-7:71.4% remission, 95.2% response.
BDI-II: 71.4% remission, 85.7% response.
PHQ-9:61.9% remission, 85.7% response.

Interestingly, while the primary focus was anxiety treatment, depression scores also showed significant improvement, supporting the notion that enhancing neuroplasticity in the DLPFC may have broad-spectrum therapeutic effects across anxiety and mood disorders.

Of note, one patient in this case series had a documented history of specific phobias, including claustrophobia and a marked fear of flying, consistent with DSM-5 criteria for Specific Phobia. Prior to treatment, the patient reported significant anticipatory anxiety and functional impairment associated with these phobias. However, within two weeks following the completion of the TMS treatment protocol, the patient began planning international travel—an activity previously avoided due to severe fear of flying. By four weeks post-treatment, the patient underwent a CT scan without distress, reporting that prior to TMS she would have required extensive mental preparation over several weeks to tolerate the confined space. Post-treatment, she experienced no significant anxiety during the imaging procedure, suggesting a meaningful reduction in phobic and generalized anxiety symptoms.

These observed changes on HAM-A, GAD-7, PHQ-9, BDI-II illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, self-awareness deficits, impaired self-regulation, impaired insight and self-control, suicidal ideation.

### Example 5

### TMS Protocol:

Two patients with Major Depressive Disorder (MDD) and Attention Deficit Hyperactivity Disorder (ADHD) were treated using a combination of 20 mg lisdexamfetamine (a stimulant-class dopaminergic medication) and 125 mg D-cycloserine (an NMDA receptor partial agonist). A single dose of each medication was taken in the morning.
60 minutes after the medication dose, patients received 20 sessions of TMS using a double-target protocol: first, they received intermittent theta-burst stimulation (ITBS) (600 pulses per session, lasting 3 minutes each). This was immediately followed by continuous theta-burst stimulation (cTBS) (600 pulses per session, lasting 33 seconds).
The TMS sessions were spaced 20 minutes apart over the course of 6 hours and 25 minutes, ensuring the total treatment time occurred within 5 half-lives of both lisdexamfetamine and D-cycloserine.
TMS was delivered using the Ampa One system. The iTBS targeted the dorsomedial prefrontal cortex, and the cTBS targeted the orbitofrontal cortex.

### Outcome Measurement:

The following instruments were used to evaluate treatment outcomes:
1. 1.Adult ADHD Self-Report Scale (ASRS) used to measure ADHD severity.
2. 9-item Patient Health Questionnaire (PHQ-9) to assess depressive symptoms.

### Results:

Patient 1:
   ASRS score decreased from 13 to 5 four weeks after treatment, indicating significant symptom improvement.
   PHQ-9 score decreased from 7 to 4 four weeks after treatment, indicating symptom remission
Patient 2:
   ASRS score decreased from 16 to 12 four weeks after treatment, indicating symptom improvement.
   PHQ-9 score decreased from 13 to 4 four weeks after treatment, indicating symptom remission

These observed changes on ASRS v1.1 illustrate meaningful therapeutic effects in attention, vigilance, sustained focus, distractibility, impulsivity, compulsivity, executive function, cognitive flexibility, problem-solving, decision-making deficits, emotional regulation deficits, motivation deficits, impaired self-regulation, impaired impulse control, social functioning deficits, interpersonal effectiveness deficits.

### Example 6

### TMS Protocol:

Two patients with Post-Traumatic Stress Disorder (PTSD) were treated using a combination of 20 mg lisdexamfetamine (a stimulant-class dopaminergic medication) and 125 mg D-cycloserine (an NMDA receptor partial agonist). A single dose of each medication was taken in the morning.
60 minutes after the medications were taken, the patient received 20 sessions of TMS using a double-target protocol: first, they received intermittent theta-burst stimulation (ITBS) (600 pulses per session, lasting 3 minutes each). This was immediately followed by continuous theta-burst stimulation (cTBS) (600 pulses per session, lasting 33 seconds).
The TMS sessions were spaced 20 minutes apart over the course of 6 hours and 25 minutes, ensuring the total treatment time occurred within 5 half-lives of both lisdexamfetamine and D-cycloserine.
TMS was delivered using the Ampa One system (coil and cap), connected to a MagVenture R30 device. The iTBS targeted the dorsomedial prefrontal cortex, and the cTBS targeted the orbitofrontal cortex.

### Outcome Measurement:

The following instrument was used to evaluate treatment outcomes:
1. PTSD Checklist for DSM-5 (PCL-5) to measure PTSD severity.

### Results:

Patient 1:
   PCL-5 score decreased from 49 to 27 three weeks after treatment, indicating significant symptom improvement and a positive response trajectory.
Patient 2:
   PCL-5 score decreased from 62 to 49 three weeks after treatment, indicating some treatment response

These observed changes on PCL-5 illustrate meaningful therapeutic effects in anxiety, stress resilience, avoidance behaviors, withdrawal, hyperarousal, hypervigilance, emotional regulation deficits, dissociative experiences, impaired self-regulation, impaired insight and self-control.

### Example 7

### “ONE-BP”

We treated 14 patients with Bipolar 1 Disorder (current episode depressed) using only 125 mg d-cycloserine (NMDA partial agonist) compounded into an oral dissolving tablet given as a single dose in the AM. Beginning 60 minutes after this dose, we then administered 20 sessions of TMS (using a pattern called intermittent theta-burst stimulation, iTBS, 600 pulses, which takes 3 minutes). Sessions were given every 20 minutes for a total of 6 hours 23 minutes of treatment time. TMS was administered on the Ampa One system (coil and cap, connected to a MagVenture R30 device), targeting the dorsomedial prefrontal cortex. Patients returned home after treatment that day, and did not receive any additional sessions thereafter.

To evaluate treatment outcomes we used 3 standardized instruments commonly used in clinical trials for measuring the severity of depression symptoms: 1) A clinician-rated scale, the 17-item Hamilton Rating Scale for Depression (HRSD); 2) A patient-rated scale, the 9-item Patient Health Questionnaire; 3) A second patient-rated scale, the 21-item Beck Depression Inventory-II. Given the bipolar diagnosis, the Young Mania Rating Scale (YMRS) was also administered, a clinician-administered tool specifically designed to assess the severity of manic symptoms. Additional assessments included the 7-item Generalized Anxiety Disorder scale (GAD-7) to capture anxiety symptoms, and the 12-item World Health Organization Disability Assessment Schedule (WHODAS 2.0) to evaluate functional impairment and overall psychosocial well-being. All instruments were administered at the baseline timepoint, defined as immediately following administration of the medication combination but prior to the initiation of treatment. These scales were then administered again in follow-up at a series of timepoints 1, 2, 3, 4, and 5 weeks after the day of treatment, and the results were plotted in FIG. 3.

The standard shape of the curve for improvement in depression symptoms with conventional once-daily TMS is an exponential decay curve, spanning several weeks (since the conventional course is 36 sessions, 5 days a week, over about 7 weeks).

### Observations:

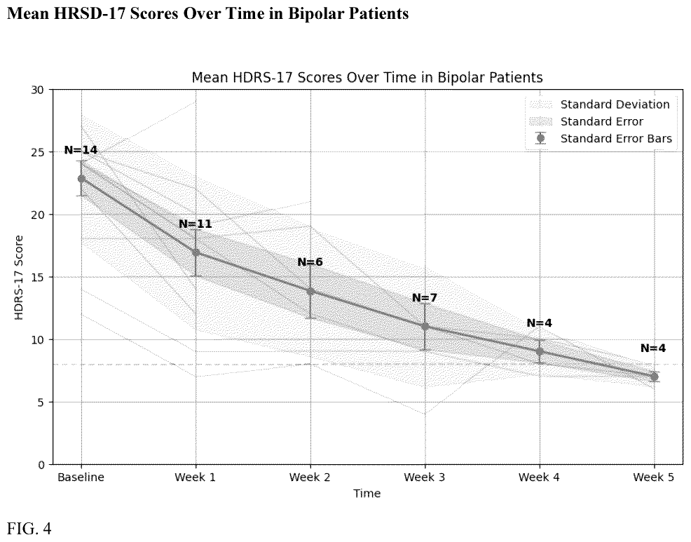

Interestingly, even though we administered all the treatments in a single day, patients showed the same exponential decay curve of improvement over the first 1-3 weeks, reaching a plateau around weeks 4-6. So this treatment regimen allowed all the sessions to be done in a single day, but only slightly accelerated the actual course of improvement. FIG. 4 plots mean HRSD-17 scores over time for the first 14 patients to complete treatment.

Bipolar disorder remains clinically challenging to treat due to the risk that interventions targeting one mood pole (e.g., depression) may inadvertently precipitate symptoms of the opposite pole (e.g., mania). In this preliminary analysis (N=4 at week 5), 21.4% of participants achieved remission and 35.7% met criteria for clinical response on the Hamilton Rating Scale for Depression (HRSD). Comparable outcomes were observed on the Patient Health Questionnaire-9 (PHQ-9), with 21.4% achieving remission and 35.7% reaching response. Importantly, no participants experienced an increase in manic symptoms; rather, mean Young Mania Rating Scale (YMRS) scores decreased from 6.3 at baseline to 1.7 at week 5. These initial findings suggest the intervention may reduce depressive symptoms without inducing manic activation. Monitoring will continue through weeks 6 and 12 to assess longer-term mood stability and treatment durability.

These observed changes on HDRS-17, PHQ-9, BDI-II, GAD-7, WHODAS, YMRS illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, executive function, cognitive flexibility, problem-solving, decision-making deficits, self-awareness deficits, impaired self-regulation, impaired insight and self-control, social functioning deficits, interpersonal effectiveness deficits, participation, mobility, life activities, suicidal ideation.

### Example 8

### “AMPLIFI”

We treated 29 patients with depression using a combination of 20 mg lisdexamfetamine (a member of the stimulant class of dopaminergic medications) with 125 mg d-cycloserine (NMDA partial agonist) compounded into a troche given as a single dose in the AM. Beginning 60 minutes after this dose, we then administered 20 sessions of TMS. Treatment was delivered every 20 minutes for a total of 6 hours 23 minutes of treatment time. Treatment targets included the dorsomedial prefrontal cortex with intermittent theta burst stimulation (3 minutes 30 seconds), followed immediately by the orbitofrontal cortex with continuous theta burst stimulation (33 seconds). TMS was administered on the Ampa One system (coil and cap, connected to a MagVenture R30 device). Patients returned home after treatment that day, and did not receive any additional sessions thereafter.

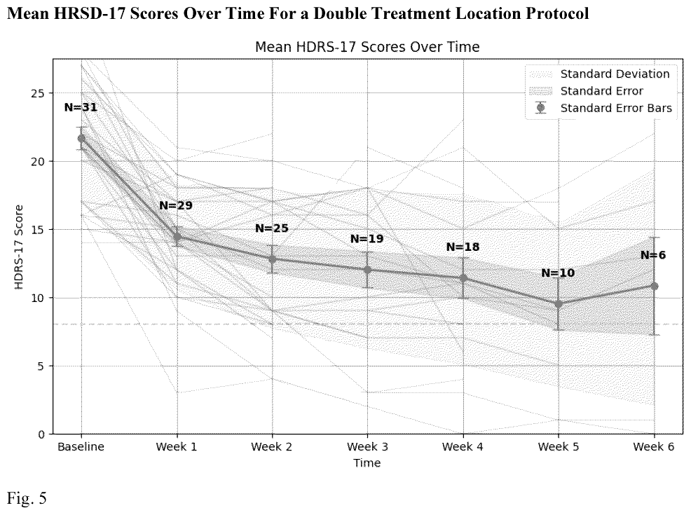

To evaluate treatment outcomes we used 3 standardized instruments commonly used in clinical trials for measuring the severity of depression symptoms: 1) A clinician-rated scale, the 17-item Hamilton Rating Scale for Depression (HRSD); 2) A patient-rated scale, the 9-item Patient Health Questionnaire; 3) A second patient-rated scale, the 21-item Beck Depression Inventory-II. Additional assessments included the 7-item Generalized Anxiety Disorder scale (GAD-7) to capture anxiety symptoms, and the 12-item World Health Organization Disability Assessment Schedule (WHODAS 2.0) to evaluate functional impairment and overall psychosocial well-being. All instruments were administered at the baseline timepoint, defined as immediately following administration of the medication combination but prior to the initiation of treatment. All scales were administered at a “baseline” timepoints immediately after giving the medication combination, but before starting treatment. These scales were then administered again in follow-up at a series of timepoints 1, 2, 3, 4, and 5, and 6 weeks after the day of treatment, and the results were plotted in FIG. 5.

Although all treatment sessions were delivered within a single day, patients still exhibited the characteristic exponential decline in depressive symptoms over time. As shown in FIG. 5, mean HRSD-17 scores dropped sharply during the first 1-2 weeks and continued to improve gradually through week 5, approaching the remission threshold (HRSD <8, marked by the red dashed line). A slight increase in mean scores was observed at week 6, likely attributable to reduced sample size (N=6) and variability in long-term response. These findings suggest that while the treatment was delivered acutely, its therapeutic effects unfolded over time in a pattern resembling that of traditional, multi-week TMS regimens.

In this preliminary analysis (N=6 at week 6), 22.6% of participants achieved remission and 38.7% met criteria for clinical response on the Hamilton Rating Scale for Depression (HRSD). Similar patterns were observed on the Patient Health Questionnaire-9 (PHQ-9), with 22.6% achieving remission and 64.5% meeting response criteria, and on the Beck Depression Inventory-II (BDI-II), with 32.3% achieving remission and 54.8% achieving response. Notably, anxiety symptoms—as measured by the Generalized Anxiety Disorder-7 (GAD-7) scale—showed the greatest relative improvement, with 41.9% reaching remission and 64.5% demonstrating response. Functional outcomes, assessed via the World Health Organization Disability Assessment Schedule (WHODAS 2.0), improved in all participants, with the average score decreasing by nearly half, from 50.2 at baseline to 26.2 at week 6. These preliminary findings suggest that the intervention may not only alleviate depressive and anxiety symptoms but also enhance overall functioning and quality of life. Given the small sample size at week 6 and the absence of data at week 12, continued monitoring is underway to evaluate the longer-term durability and stability of treatment effects.

These observed changes on HDRS-17, PHQ-9, BDI-II, GAD-7, WHODAS illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, executive function, cognitive flexibility, problem-solving, decision-making deficits, self-awareness deficits, impaired self-regulation, impaired insight and self-control, social functioning deficits, interpersonal effectiveness deficits, participation, mobility, life activities, suicidal ideation.

### Example 9

### TMS Protocol:

Two patients with Alzheimer's were treated using 125 mg D-cycloserine (an NMDA receptor partial agonist). A single dose of the medication was taken in the morning.
60 minutes after the medications were taken, the patient received 20 sessions of TMS using a single-target protocol: intermittent theta-burst stimulation (ITBS) (600 pulses per session, lasting 3 minutes each).
The TMS sessions were spaced 20 minutes apart over the course of 6 hours and 25 minutes, ensuring the total treatment time occurred within 5 half-lives of the D-cycloserine.
TMS was delivered using the Ampa One system (coil and cap), connected to a MagVenture R30 device. The iTBS targeted the precuneus region.

### Outcome Measurement:

The following instrument was used to evaluate treatment outcomes:
1. Alzheimer's Disease Assessment Scale-Cognitive Subscale-13 (ADAS-COG13): used to assess cognitive function and detect cognitive impairment
2. Montreal Cognitive Assessment (MoCA): used to assess cognitive function and detect cognitive impairment
3. Functional Activity Questionnaire (FAQ): used to measure instrumental activities of daily living

### Results:

Patient 1:
   ADAS-COG13 score decreased from 30.7 to 25.3 three weeks after treatment, indicating significant symptom improvement.
   MoCA score improved from 8 to 13, indicating moderate improvement.
Patient 2:
   ADAS-COG13 score decreased from 24 to 17.7 two weeks after treatment, indicating significant symptom improvement.
   MoCA score decreased slightly from 16 at baseline to 14 at two weeks post-treatment, suggesting relatively stable cognitive performance over this period. However, it is important to interpret these results with caution, as the patient has completed MoCA assessments on a near-weekly basis for the past three years, which may reduce the measure's sensitivity due to familiarity effects.

These observed changes on ADAS-COG, MOCA illustrate meaningful therapeutic effects in cognitive function, cognition, memory, learning, attention, vigilance, sustained focus, distractibility, executive function, cognitive flexibility, problem-solving, decision-making, language, orientation, self-awareness, insight, judgment.

### Example 10

We enrolled four adults with mixed anxiety and depressive symptoms, with baseline PHQ-9 scores ranging from 6 to 27 and GAD-7 scores ranging from 10 to 21. Each participant received a total of 3 grams of sarcosine daily, administered as 2 grams in the morning one hour before treatment, and an additional 1 gram after the eighth TMS session, spaced 20 minutes apart. This dosing continued for 4 to 6 weeks of follow-up.

Sixty minutes after the initial dose, participants underwent 20 sessions of transcranial magnetic stimulation (TMS) in a single day. Sessions were spaced 20 minutes apart. Each session consisted of intermittent theta-burst stimulation (iTBS) delivering 600 pulses over approximately 3 minutes. The total treatment time was about 6 hours and 25 minutes. Stimulation targeted the dorsomedial prefrontal cortex using the Brainsway H7 device. The entire stimulation protocol was completed within five half-life periods of sarcosine.

No additional TMS sessions or changes in medication were made during the follow-up period.

Outcomes were assessed weekly for 4 to 6 weeks using patient-rated instruments: the Patient Health Questionnaire-9 (PHQ-9) for depressive symptoms and the Generalized Anxiety Disorder-7 (GAD-7) scale for anxiety symptoms.

Patient symptom scores by week:

### Patient JB:

$$\begin{matrix}Baseline : PHQ - 9 = 27 , GAD - 7 = 19 \\ Week 1 : PHQ - 9 = 23 , GAD - 7 = 15 \\ Week 2 : PHQ - 9 = 16 , GAD - 7 = 14 \\ Week 3 : PHQ - 9 = 13 , GAD - 7 = 9 \\ Week 4 : PHQ - 9 = 12 , GAD - 7 = 10\end{matrix}$$

### Patient EB:

$$\begin{matrix}Baseline : PHQ - 9 = 6 , GAD - 7 = 16 \\ Week 1 : PHQ - 9 = 6 , GAD - 7 = 7 \\ Week 2 : PHQ - 9 = 5 , GAD - 7 = 5 \\ Week 3 : PHQ - 9 = 6 , GAD - 7 = 6 \\ Week 4 : PHQ - 9 = 5 , GAD - 7 = 7\end{matrix}$$

### Patient JS:

$$\begin{matrix}Baseline : PHQ - 9 = 27 , GAD - 7 = 21 \\ Week 2 : PHQ - 9 = 23 , GAD - 7 = 21 \\ Week 3 : PHQ - 9 = 14 , GAD - 7 = 19 \\ Week 4 : PHQ - 9 = 11 , GAD - 7 = 7 \\ Week 5 : PHQ - 9 = 14 , GAD - 7 = 12 \\ Week 6 : PHQ - 9 = 15 , GAD - 7 = 19\end{matrix}$$

### Patient TB:

$$\begin{matrix}Baseline : PHQ - 9 = 20 , GAD - 7 = 10 \\ Week 1 : PHQ - 9 = 15 , GAD - 7 = 8 \\ Week 2 : PHQ - 9 = 15 , GAD - 7 = 9 \\ Week 3 : PHQ - 9 = 12 , GAD - 7 = 9 \\ Week 4 : PHQ - 9 = 11 , GAD - 7 = 7\end{matrix}$$

These observed changes on the PHQ-9 and GAD-7 illustrate meaningful therapeutic effects in anxiety, stress resilience, worry, rumination, fatigue, energy deficits, low alertness, low arousal, motivation deficits, apathy, loss of initiative, emotional regulation deficits, anhedonia, reduced emotional well-being, psychological flourishing, sleep architecture disturbances, cognitive function, memory, concentration, self-awareness deficits, impaired self-regulation, impaired insight and self-control, and suicidal ideation, consistent with the functional performance domains described in the patent.

### Example 11

Based on the results reported in Examples 1-10, patients with various other psychiatric and neurological disorders can be treated using different types of neuromodulation+different neuroplasticity-enhancing agents, such as, but not limited to the following combinations:

| | | Neuroplasticity-Enhancing |
|---|---|---|
| Indication | Neuromodulation | Agent |
| Depression | TMS | Donepezil + D-cycloserine |
| Depression | TMS | Guanfacine |
| Depression | TMS | Sarcosine + Vyvanse |
| Binge eating | | |
| disorder | TMS | D-cycloserine + Vyvanse |
| Anorexia | TMS | D-cycloserine + Vyvanse |
| Nervosa | | |
| Insomnia | | |
| Disorder | TMS | D-cycloserine + Vyvanse |
| Substance Abuse | TMS | D-cycloserine |
| or Dependence | | |
| Impulse Control | TMS | D-cycloserine + Vyvanse |
| Disorders | | |
| Migraine | TMS | D-cycloserine + Vyvanse |
| Stroke | TMS | D-cycloserine + Vyvanse |
| Parkinson's | TMS | D-cycloserine + Vyvanse |
| Depression | Transcranial Electrical | D-cycloserine |
| | Stimulation (TES) | |
| Anxiety | Transcranial Electrical | D-cycloserine |
| | Stimulation (TES) | |
| Insomnia | Transcranial Electrical | D-cycloserine |
| | Stimulation (TES) | |
| PTSD | Transcranial Electrical | D-cycloserine |
| | Stimulation (TES) | |
| Parkinson's | Focused Ultrasound | D-cycloserine |
| Anxiety | Focused Ultrasound | D-cycloserine |
| Depression | Focused Ultrasound | D-cycloserine |
| Depression | Electroconvulsive | D-cycloserine |
| | Therapy (ECT) | |

### Example 12

### Patients and Treatments:

A single-day course of 30 sessions of intermittent theta-burst TMS (ITBS) is combined with L-DOPA (Levodopa), a dopamine precursor commonly used for treating Parkinson's disease.
L-DOPA is administered orally at a dose of 100 mg, 30 minutes prior to the initiation of TMS.

### TMS Protocol:

The TMS sessions are delivered every 30 minutes over the course of the day.
TMS is targeted at the motor cortex and other brain regions affected by Parkinson's disease, aiming to reorganize neural circuits to improve motor function.

### Pharmacological Action:

L-DOPA is metabolized to dopamine in the brain, increasing dopamine availability and enhancing dopaminergic transmission. This promotes neuroplasticity, making the TMS sessions more effective by boosting the brain's capacity to reorganize neural pathways affected by Parkinson's disease.

### Outcome:

The combination of L-DOPA and TMS reduces motor symptoms more effectively than TMS alone by enhancing neuroplasticity through dopamine modulation.
This approach also has benefits in treating depression and other mood disorders, due to the positive impact of increased dopamine levels on mood regulation.

### Example 13

### Patients and Treatment:

A single-day course of 20 sessions of transcranial direct current stimulation (TDCS) is combined with Aniracetam, a positive allosteric modulator of AMPA receptors, which enhances glutamatergic transmission.
Aniracetam is administered orally at a dose of 750 mg, 1 hour before the initiation of the TDCS sessions.

### TDCS Protocol:

TDCS is applied to brain regions involved in cognitive function, including the prefrontal cortex and parietal cortex. These regions are critical for memory formation and cognitive processing, particularly in the treatment of cognitive impairment or neurodegenerative diseases such as Alzheimer's disease.

### Pharmacological Action:

Aniracetam activates AMPA receptors, promoting synaptic plasticity by increasing calcium influx and enhancing long-term potentiation (LTP), a process crucial for memory formation and cognitive function.
The potentiation of AMPA receptor activity by Aniracetam complements TDCS by promoting stronger and more durable synaptic connections, thus enhancing the overall effectiveness of the stimulation.

### Outcome:

The combined approach of Aniracetam and TDCS reduces the number of TDCS sessions required to achieve cognitive enhancement and improve long-term outcomes in patients.
This method is particularly relevant for treating patients with Alzheimer's disease or other neurocognitive disorders, where enhancing synaptic plasticity may lead to significant cognitive improvements.

### Example 14

In this example, transcranial magnetic stimulation (TMS) is used with D-cycloserine (DCS) and Bupropion to treat anxiety disorder. The patient undergoes 20 TMS sessions in one day, spaced 45 minutes apart, with targeted stimulation at scalp location AF8, known for regulating anxiety.

DCS is administered at 50 mg, 30 minutes before the first session to enhance NMDA receptor-driven neuroplasticity. It is re-dosed with another 50 mg halfway through the day to maintain effectiveness. Bupropion (150 mg) is taken 30 minutes before the second TMS session to increase dopamine and norepinephrine levels, further boosting neuroplasticity.

Throughout the treatment, the patient completes the Hamilton Anxiety Rating Scale (HAM-A) (clinician-rated) and the Beck Anxiety Inventory (BAI) (patient-rated) before and after the sessions to measure changes in anxiety severity. The combined pharmacological and TMS approach expedites symptom reduction, with continuous monitoring ensuring treatment effectiveness.

### Example 15

In this example, focused ultrasound (FUS) is combined with D-Cycloserine and Pramipexole to treat adolescents (ages 12 to 17) with Antisocial Personality Disorder (ASPD). The protocol involves 8 TMS sessions per day for 5 consecutive weekdays (40 total sessions).

D-Cycloserine (DCS), an NMDA receptor partial agonist, is administered at a dose of 50 mg orally, 30 minutes before the first FUS session of each day. DCS enhances synaptic plasticity by facilitating glutamatergic transmission, aiding in behavioral and emotional regulation in adolescents with ASPD.

Pramipexole, a dopamine receptor agonist (D2/D3), is administered at a dose of 0.25 mg, 30 minutes before the first FUS session each day. Pramipexole enhances dopaminergic transmission, promoting neuroplasticity and improving emotional regulation and impulse control—critical areas of difficulty for individuals with ASPD.

The TMS sessions target the dorsolateral prefrontal cortex (DLPFC), an area associated with impulse control and emotional regulation. Each TMS session lasts 3-10 minutes, with 90 minute intervals between treatments.

Progress is measured using the Youth Psychopathic Traits Inventory (YPI) (clinician-rated) and Strengths and Difficulties Questionnaire (SDQ) (self-rated) to assess changes in antisocial behaviors and emotional regulation before and for weeks after treatment.

### Example 16

### Patients and Treatment:

A course of 5 separate treatment sessions combining Psilocybin and D-Cycloserine (DCS) is used to treat depression. Each session occurs on a different day, spaced over several weeks to allow for reflection and integration between sessions.

### Psilocybin Administration:

Psilocybin is administered at a moderate dose of 25 mg under controlled, therapeutic conditions. As a 5-HT2A receptor agonist, Psilocybin induces significant alterations in consciousness, facilitating emotional and cognitive insights that are therapeutic for depression. Its neuroplastic effects help reset brain circuits related to mood regulation, enabling more flexible thought patterns and emotional processing.

### D-Cycloserine Administration:

D-Cycloserine (DCS) is administered orally at a dose of 50 mg, 30 minutes before each Psilocybin session. DCS enhances neuroplasticity by facilitating NMDA receptor activity, which is key to synaptic strengthening and consolidating the therapeutic insights gained during the Psilocybin experience. This combination is designed to help patients integrate emotional and cognitive breakthroughs more effectively, leading to longer-lasting improvements in depressive symptoms.

### Treatment Setting:

Each session is conducted in a controlled environment with therapeutic support, ensuring the safe exploration of Psilocybin's effects. Between sessions, patients engage in reflection and integration practices, such as journaling, mindfulness, or therapy, to process and consolidate the insights from the psychedelic experience.

### Outcome Measurement:

Progress is measured using the Hamilton Depression Rating Scale (HDRS) (clinician-rated) and the Beck Depression Inventory (BDI) (patient-rated), both administered before and after each session to track changes in depressive symptoms and overall emotional well-being.

1. A treatment protocol for treating a condition of the central nervous system (CNS) in an individual in need of treatment, wherein the condition comprises a condition selected from the group consisting of an anxiety disorder; a stress disorder; a psychotic disorder; a personality disorder; an impulse-control disorder; an addiction disorder; an eating disorder; a sleep disorder; a sexual disorder; a neurodevelopmental disorder; a neurological disorder; and a functional performance domain; the treatment protocol comprising administering to the individual at least one neuroplasticity-potentiating pharmaceutical agent, and delivering a multi-session energy-based neuromodulation treatment to said individual over multiple sessions within a time period equal to or less than five half-life time periods of the at least one neuroplasticity-potentiating pharmaceutical agent, of the time of administration of the at least one neuroplasticity-potentiating pharmaceutical agent to said individual.

2. The treatment protocol of claim 1, wherein said energy-based neuromodulation treatment comprises at least one energy-based neuromodulation treatment selected from the group consisting of transcranial magnetic stimulation (TMS); transcranial electrical stimulation; convulsive neuromodulation; energy-based focal stimulation; implantable bioelectric stimulation; and genetically-targeted neuromodulation.

3. The treatment protocol of claim 1, wherein the neuroplasticity-potentiating pharmaceutical agent comprises at least one agent selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator.

4. The treatment protocol of claim 1, wherein said energy-based neuromodulation treatment comprises transcranial magnetic stimulation (TMS), and wherein the neuroplasticity-potentiating pharmaceutical agent comprises an NMDA receptor agonist, optionally administered in combination with a dopaminergic agent.

5. The treatment protocol of claim 1, characterized by comprising one or more of the following conditions: (a) said energy-based neuromodulation is delivered at less than 80% of an individualized therapeutic threshold; (b) a dominant pulse frequency is 5 Hz or less; (c) a treatment session duration is 30 minutes or less; and (d) a total number of therapeutic pulses per session is fewer than 600.

6-17. (canceled)

18. The treatment protocol of claim 1, wherein the multiple sessions of said multi-session energy-based neuromodulation treatment are delivered before, during or after administration of the at least one neuroplasticity-potentiating pharmaceutical agents.

19. The treatment protocol of claim 1, wherein the neuroplasticity-potentiating pharmaceutical agent comprises an NMDA agonist/partial agonist.

20. A method for enhancing and/or accelerating a therapeutic effect of a multi-session energy-based neuromodulation treatment of the central nervous system (CNS) in an individual to treat a condition selected from the group consisting of an anxiety disorder; a stress disorder; a psychotic disorder; a personality disorder; an impulse-control disorder; an addiction disorder; an eating disorder; a sleep disorder; a sexual disorder; a neurodevelopmental disorder; a neurological disorder; and a functional performance domain; the method comprising administering to the individual (1) at least one neuroplasticity-potentiating pharmaceutical agent selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator, and (2) a dopamine agent, and delivering said energy-based neuromodulation treatment of said individual in a series of sessions beginning within a time period less than a five half-life time period of said at least one neuroplasticity-potentiating pharmaceutical agent and said dopamine agent after administering the at least one of the neuroplasticity-potentiating pharmaceutical agent and the dopamine agent.

21. The method of claim 20, wherein said energy-based neuromodulation treatment comprises at least one treatment method selected from the group consisting of transcranial magnetic stimulation (TMS); transcranial electrical stimulation; convulsive neuromodulation; energy-based focal stimulation; implantable bioelectric stimulation; and genetically-targeted neuromodulation.

22. The method of claim 20, characterized by comprising one or more of the following conditions: (a) said energy-based neuromodulation treatment is delivered at less than 80% of an individualized therapeutic threshold; (b) a dominant pulse frequency is 5 Hz or less; (c) a treatment session duration is 30 minutes or less; and (d) a total number of therapeutic pulses per session is fewer than 600.

23. A method for enhancing and/or accelerating a therapeutic effect of an energy-based multi-session neuromodulation treatments of the central nervous system (CNS) in an individual to treat a condition selected from the group consisting of an anxiety disorder; a stress disorder; a psychotic disorder; a personality disorder; an impulse-control disorder; an addiction disorder; an eating disorder; a sleep disorder; a sexual disorder; a neurodevelopmental disorder; a neurological disorder; and a functional performance domain; the method comprising administering to the individual at least one two pharmaceutical agents including (1) at least neuroplasticity-potentiating pharmaceutical agent, selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator; and (2) a dopaminergic agent, wherein said at least one neuroplasticity-potentiating agent and said dopaminergic agent are administered to said individual during or between said multi-session treatments of said individual.

24. The method of claim 23, wherein said energy-based neuromodulation treatment comprises at least one treatment method selected from the group consisting of transcranial energy-based focal stimulation; implantable bioelectric stimulation; and genetically-targeted neuromodulation.

25. The method of claim 23, wherein said energy-based neuromodulation treatment comprises transcranial magnetic stimulation (TMS), and wherein the neuroplasticity-potentiating pharmaceutical agent comprises an NMDA receptor agonist, administered with a dopaminergic agent.

26. The method of claim 23, characterized by comprising one or more of the following conditions: (a) said energy-based neuromodulation treatment is predominantly delivered at less than 80% of an individualized therapeutic threshold; (b) a dominant pulse frequency is 5 Hz or less; (c) a treatment session duration is 30 minutes or less; and (d) a total number of therapeutic pulses per session is fewer than 600.

27. A method for enhancing and/or accelerating a therapeutic effect of an energy-based neuromodulation multi-session treatments of the central nervous system (CNS) in an individual to treat a condition selected from the group consisting of an anxiety disorder; a stress disorder; a psychotic disorder; a personality disorder; an impulse-control disorder; an addiction disorder; an eating disorder; a sleep disorder; a sexual disorder; a neurodevelopmental disorder; a neurological disorder; and a functional performance domain; the method comprising delivering said energy-based neuromodulating treatment to said individual, and administering to the individual at least two pharmaceutical agents including (1) at least one neuroplasticity-potentiating pharmaceutical agent, selected from the group consisting of an N-methyl-D-aspartate (NMDA) receptor modulator; a non-NMDA glutamatergic modulator; a monoaminergic modulator; a cholinergic modulator; a gamma-aminobutyric acid (GABAergic) modulator; an opioidergic modulator; an endocannabinoid modulator; a cellular signaling modulator; an epigenetic modulator; a transcriptional modulator; a structural modulator; a synaptic modulator; a neurotrophic modulator; a neuroimmune modulator; and a glial modulator; and (2) a dopaminergic agent, wherein said at least one neuroplasticity-potentiating agent and said at least one dopamine agent are administered to said individual following said multi-session treatment of said individual.

28. The method of claim 27, wherein said energy-based neuromodulation treatment comprises at least one treatment method selected from the group consisting of transcranial energy-based focal stimulation; implantable bioelectric stimulation; and genetically-targeted neuromodulation.

29. The method of claim 27, wherein said energy-based neuromodulation treatment comprises transcranial magnetic stimulation (TMS), and wherein the neuroplasticity-potentiating pharmaceutical agent comprises an NMDA receptor agonist, optionally administered in combination with a dopaminergic agent.

30. The method of claim 27, characterized by comprising one or more of the following conditions: (a) said energy-based neuromodulation treatment is predominantly delivered at less than 80% of an individualized therapeutic threshold; (b) a dominant pulse frequency is 5 Hz or less; (c) a treatment session duration is 30 minutes or less; and (d) a total number of therapeutic pulses per session is fewer than 600.
